(19) United States
(12) Patent Application Publication
(10) Pub. No.: US 2004/0142317 A1
(43) Pub. Date: Jul. 22, 2004

(54) Use of amino acid transporter atbo,+ as a delivery system for drugs and prodrugs

(76) Inventors: Vadivel Ganapathy, Martinez, GA (US); Takahiro Hatanaka, Shizuoka (JP)

Correspondence Address: FOLEY AND LARDNER, SUITE 500, 3000 K STREET NW, WASHINGTON, DC, 20007, US

(21) Appl. No.: 10/467,893

(22) PCT Filed: Apr. 12, 2002
(86) PCT No.: PCT/JP02/03678

(51) Int. Cl. C12Q 1/00; G01N 33/53; G01N 33/567

(52) U.S. Cl. 435/4; 435/7.2

(57) ABSTRACT

The present invention has revealed the compounds transportable by ATB⁰⁺. Based on the information about these compounds, drugs transportable by ATB^{0,+} may be designed, produced and screened. Such drugs may serve to treat and/or prevent the diseases in which NOS, phenylglycine, carnitine, D-amino NOS, phenylglycine, carnivolved. The ATB^{0,+} gene may be administered to patients to be used for gene therapy of the diseases as described above.

### TECHNICAL FIELD

The present invention relates to the use of amino acid transporter ATB^{0,+} as a delivery system for drugs and prodrugs.

### BACKGROUND ART

Nitric oxide (NO) is an important regulatory molecule involved in a variety of physiological processes (Moncada, S., J.R.Soc.Med., 92, 164-169, (1999), Martin, E. et al., Sem.Perinatol., 24, 2-6, (2000), Bredt, D. S., Free Rad.Res. 31, 577-596, (1999)). This molecule is generated from L-arginine by nitric oxide synthases (NOS). Three distinct isoforms of NOS have been identified: neuronal NOS (nNOS or NOS I), inducible NOS (iNOS or NOS II), and endothelial NOS (eNOS or NOS III) (Knowles R. G., and Moncada, S. Biochem.J., 298, 249-258, (1994), Stuehr, D. J., Biochim.Biophys.Acta, 1411, 217-230, (1999)). Even though NO plays an essential role in many physiological processes, overproduction of NO is associated with a multitude of pathological conditions including inflammation, septic shock, diabetes, and neurodegeneration (Miller, M. J. S. and Grisham, M. B., Mediators inflammation, 4, 387-396, (1995), Symeonides, S. and Balk, R. A., Infect.Dis.Clin.N.Amer., 13, 449-463, (1999), Mandrup-Poulsen, T., Diabetologia, 39, 1005-1029, (1996), Jenner, P. and Olanow, C. W., Neurology, 47, S161-S170, (1996)). Blockade of NO production by inhibition of NOS may therefore have potential in the treatment of these pathological conditions. Since different isoforms of NOS are involved in different pathological conditions, selective inhibition of specific isoforms of NOS will become necessary to enhance the therapeutic use of this approach for differential treatment of these disorders. Several inhibitors have been identified that are selective for different NOS isoforms (Southan, G. J. and Szabo, C., Biochem.Pharmacol., 51, 383-394, (1996), Bryk, R. and Wolff, D. J., Pharmacol:Ther., 84, 157-178, (1999)). Use of these inhibitors has been shown to be beneficial in the treatment of diverse conditions associated with overproduction of NO in humans and in experimental animals (Moncada, S. and Higgs, E. A., FASEB J., 9, 1319-1330, (1995), Hobbs, A. J. et al., Annu.Rev.Pharmacol.Toxicol., 39, 191-220, (1999)).

The therapeutic efficacy of NOS inhibitors is expected to be influenced markedly by the efficiency with which these inhibitors are taken up into the target cells for interaction with NOS. Furthermore, transport of these inhibitors in the intestine will influence their oral bioavailability. Therefore, information on the mechanisms of cellular uptake of NOS inhibitors is critical to assess their therapeutic potential. Most NOS inhibitors are structurally related to arginine, lysine, citrulline, and ornithine (Southan, G. J. and Szabo, C., Biochem.Pharmacol., 51, 383-394, (1996), Bryk, R. and Wolff, D. J., Pharmacol.Ther., 84, 157-178, (1999)). Consequently, amino acid transport systems play a critical role in the cellular uptake of NOS inhibitors. Multiple systems operate in mammalian cells to mediate the transport of amino acids and these transport systems differ markedly in substrate specificity, substrate affinity, driving forces, and tissue expression pattern (Christensen, H. N., Methods Enzymol., 173, 576-616, (1989)). Many of these transport systems have been recently cloned and functionally characterized (Palacin, M. et al., Physiol.Rev., 78, 969-1054, (1998), Ganapathy, V. et al., Intestinal transport of peptides and amino acids. In Current Topics in Membranes. Ed. Barrett, K. E. and Donowitz, M., Vol.50, pp.379-412. Academic Press. (2001)). There have been several studies in the past aimed at identifying the amino acid transport systems that mediate the uptake of NOS inhibitors (Schmidt, K. et al., Mol.Pharmacol., 44,615-621, (1993), Schmidt, K. et al., Biochem.J., 301, 313-316, (1994), Baydoun, A. R. and Mann, G. E., Biochem.Biophys.Res.Commun., 200, 726-731, (1994), Schmidt, K. et al., J.Neurochem., 64, 1469-1475, (1995), Raghavendra Rao, V. L. and Butterworth, R. F., J.Neurochem., 67, 1275-1281, (1996)). Two amino acid transport systems have so far been identified that are involved in the cellular uptake of NOS inhibitors. These are system y⁺ and system L. Both are Na⁺-independent transport systems and therefore exhibit only a weak capacity to concentrate their substrates including the NOS inhibitors inside the cells. To our knowledge, no other amino acid transport system has been shown to be involved in the transport of NOS inhibitors.

Carnitine (β-hydroxy-γ-trimethylaminobutyrate) is an obligate requirement for β-oxidation of long-chain fatty acids. It is synthesized endogenously in humans in the liver and kidney (Carter, A. L. et al., Journal of Child Neurology, 10 (supplement 2), S3-S7, (1995)). It is also absorbed in the intestinal tract from dietary sources (Rebouche, C. J., FASEB Journal, 6, 3379-3386, (1992)). The biological importance of this molecule is evident from the clinical consequences of carnitine deficiency encountered in a variety of genetic and acquired diseases (Kerner, J. and Hoppel, C., Annual Review of Nutrition, 18, 179-206, (1998)). The symptoms of carnitine deficiency include skeletal myopathy, cardiomyopathy, encephalopathy and failure to thrive (Kerner, J. and Hoppel, C., Annual Review of Nutrition, 18, 179-206, (1998); Treem, W. R. et al., New England Journal of Medicine, 319, 1331-1336, (1988). Most tissues, including the cardiac and skeletal muscle, contain intracellular carnitine levels several-fold higher than plasma levels due to the presence of a Na⁺-dependent high-affinity carnitine transport system (Bremer, J., Physiological Reviews, 63, 1420-1480, (1983)). This transport system also exists in the brush border membrane of renal tubular epithelial cells where it plays a role in the reabsorption of carnitine (Rebouche, C. J. and Mack, D. L., Archives of Biochemistry and Biophysics, 235, 393-402, (1984); Huang, W. et al., Biochemical Pharmacology, 58, 1361-1370, (1999)). A genetic defect in this transport system results in excessive urinary loss of carnitine, causing systemic carnitine deficiency. Since the same transport system is also responsible for active accumulation of carnitine in tissues such as the heart and skeletal muscle, the genetic defect is associated with drastically reduced intracellular levels of carnitine in these tissues. The major clinical symptoms of this defect, known as primary carnitine deficiency, are skeletal and cardiac myopathy, resulting from impaired energy production from fatty acid oxidation as a consequence of reduced intracellular levels of carnitine. Recently, this transporter has been cloned (Wu, X. et al., Biochemical and Biophysical Research Communications, 246, 589-595, (1998), Tamai, I. et al., Journal of Biological Chemistry, 273, 20378-20382, (1998)). Interestingly, this transporter also transports several organic cations (Wu, X. et al., Biochemical and Biophysical Research Communications, 246, 589-595, (1998), Wu, X., et al., Journal of Pharmacology and Experimental Therapeutics, 290, 1482-1492, (1999)). Furthermore, it belongs to the organic cation transporter gene family on the basis of its primary structure (Wu, X. et al., Biochemical and Biophysical Research Communications, 246, 589-595, (1998)). Therefore, the transporter is named OCTN2 (novel organic cation transporter 2).

D-Amino acids are generally considered foreign to metabolic pathways in mammals. Almost all mammalian enzymes are selective for L-amino acids with the notable exception of D-amino acid oxidase. D-Amino acids are found in the plasma, but they are believed to originate from the diet or from the intestinal microbial flora. The serum levels of most D-amino acids are reduced markedly in germ-free laboratory animals, indicating that intestinal microorganisms do contribute significantly to D-amino acids present in the serum (J. J. Corrigan, Science, 164, 142 (1969), M. Friedman, J.Agric.Food Chem., 47, 3457(1999)). Diet also contains D-amino acids that are produced from the L-enantiomers during food preparation and processing (E. H. Man, and J. L. Bada, Annu.Rev.Nutr., 7, 209 (1987)).

Since D-amino acids do not participate in metabolic pathways in mammals, the biological significance of these amino acids has remained questionable. There is evidence however for beneficial effects of certain D-amino acids (M. Friedman, J.Agric.Food Chem., 47, 3457 (1999), E. H. Man, and J. L. Bada, Annu.Rev.Nutr., 7, 209(1987)). D-Phenylalanine and D-leucine have been shown to be analgesic and have been used pharmacologically in the management of intractable pain (R. S. S. Cheng, and B. Pomeranz, Brain Res., 177, 583 (1979), K. Budd, Adv.Pain Res.Ther., 5, 305 (1983)). Similarly, a recent study has shown that administration of D-serine to schizophrenic patients may improve the cognitive function and performance (G. Tsai, et al., Biol.Psychiatry, 44,1081 (1998)). There is also evidence of a physiological function for D-serine in the modulation of glutamatergic neurotransmission (T. Matsui, et al., J.Neurochem., 65, 454 (1995), J.-P. Mothet, et al., Proc.Natl.Acad.Sci.USA, 97, 4926 (2000)). This amino acid binds to the glycine site on the N-methyl-D-aspartate receptor and this binding is obligatory for the activation of the receptor-associated Ca²⁺-channel function by glutamate. Interestingly, this D-amino acid is generated endogenously in the brain by recemization of L-serine mediated by serine recemase (H. Wolosker, et al., Proc.Natl.Acad.Sci.USA, 96, 721 (1999)). This enzyme has been cloned recently from the brain and is found to colocalize with the NMDA receptor (H. Wolosker, et al., Proc.Natl.Acad.Sci.USA, 96, 13409 (1999)).

D-Amino acids are abundant in bacteria (M. Friedman, J.Agric.Food Chem., 47, 3457 (1999)) and therefore are expected to be present in significant quantities in the colon and ileum where bacterial colonization is prevalent under physiological conditions. As mentioned earlier, diet also contains D-amino acids. Since some of the D-amino acids are now known to possess important physiological and pharmacological functions, the issue of intestinal absorption of bacteria-derived and dietary D-amino acids becomes important. If there are effective mechanisms in the intestinal tract for the entry of lumenal D-amino acids into blood, it is potentially possible for exogenous D-amino acids to exert biological effects. Several transporters that mediate the intestinal absorption of amino acids have been recently cloned (V. Ganapathy, et al., Current Topics in Membranes, 50, 379 (2001)). To date, only two amino acid transporters have thus far been shown to transport D-amino acids to any significant extent (Y. Kanai, et al., J.Biol.Chem., 273, 23629 (1998), Y. Fukasawa, et al., J.Biol.Chem., 275, 9690 (2000)). They are system L1 and system asc1. Both are facilitative transporters that mediate amino acid exchange across the plasma membrane. They are not active because their transport function is not coupled to any driving force. System L1 and system asc1 have been cloned from the brain, and transcripts for these transporters are detectable in the intestinal tract. Interestingly, these transporters are heterodimeric, both containing the heavy chain of the 4F2 cell surface antigen (4F2hc) as a common subunit. Since 4F2hc is localized exclusively to the basolateral membrane of the mucosal cells of the intestinal tract, system L1 and system asc1 are likely to participate in the efflux of amino acids from the cells into the blood. It is not known at present if there are transport systems in the brush border membrane of the intestinal epithelial cells that might mediate the entry of D-amino acids from the lumen into the cells.

The transporter, known as ATB^{0,+}, is an amino acid transporter expressed in the intestine, lung, and mammary gland. Functionally, ATB^{0,+} is a Na⁺- and Cl⁻-coupled transport system for neutral and cationic amino acids. It plays an important role in the absorption of amino acids in the intestinal tract (Ganapathy, V. et al., Intestinal transport of peptides and amino acids. In, Current Topics in Membranes (eds. Barrett, K. E. and Donowitz, M.), Vol.50, pp.379-412. Academic Press., (2001)). The cloning of human ATB^{0,+} has been recently reported (Sloan, J. L. and Mager, S., Journal of Biological Chemistry, 274, 23740-23745, (1999)). To date, the transport function of ATB^{0,+} has been studied only with amino acids as substrates. Its transport function is highly concentrative, energized by transmembrane gradients of Na⁺ and Cl⁻ and membrane potential. ATB^{0,+} belongs to the gene family of Na⁺- and Cl⁻ coupled transporters for a variety of compounds such as amino acids (e.g., glycine and proline), neurotransmitters (e.g., monoamines and γ-aminobutyrate), and osmolytes (e.g., taurine and betaine). Structurally, ATB^{0,+} is very closely related to γ-aminobutyrate transporters and betaine transporter.

### DISCLOSURE OF INVENTION

The present inventors have cloned the Na⁺- and Cl⁻-coupled amino acid transporter ATB^{0,+} from mouse colon and investigated its ability to transport NOS inhibitors. When expressed in mammalian cells, ATB^{0,+} is able to transport a variety of zwitterionic and cationic amino acids in a Na⁺- and Cl⁻-coupled manner. The present inventors tested several NOS inhibitors for their ability to inhibit ATB^{0,+}-mediated uptake of glycine and found all of them to compete with glycine for the uptake process. With [³H]-N^{ω}-nitro-L-arginine, the present inventors have demonstrated directly the Na⁺- and Cl⁻-coupled transport of this NOS inhibitor via ATB^{0,+}. We then studied the ATB^{0,+}-mediated transport of a wide variety of NOS inhibitors using the X. laevis oocyte system. This was done by assessing the NOS inhibitor-induced inward currents under voltage clamp conditions in oocytes expressing the cloned ATB^{0,+}. These studies showed that ATB^{0,+} is able to transport a broad range of zwitterionic as well as cationic NOS inhibitors. These data represent the first identification of an ion gradient-driven concentrative transport system for NOS inhibitors in the intestinal tract.

The present inventors cloned this transporter from mouse colon and expressed functionally in mammalian cells and Xenopus laevis oocytes to investigate the interaction of carnitine and its acyl esters with the transporter.

When expressed in mammalian cells, the cloned ATB^{0,+} was able to transport carnitine, propionylcarnitine, and acetylcarnitine. The transport process was Na⁺- and Cl⁻-dependent. The Michaelis-Menten constant for carnitine was 0.83±0.08 mM and the Hill coefficient for Na⁺ activation was 1.6±0.1.

When expressed in Xenopus laevis oocytes, the cloned ATB^{0,+} was able to induce inward currents in the presence of carnitine and propionylcarnitine under voltage-clamped conditions. There was no detectable current in the presence of acetylcarnitine. Carnitine-induced currents were obligatorily dependent on the presence of Na⁺ and Cl⁻. The currents were saturable with carnitine and the Michaelis-Menten constant was 1.8±0.4 mM. The analysis of Na⁺- and Cl⁻-activation kinetics revealed that 2 Na⁺ and 1 Cl⁻ were involved in the transport of carnitine via the transporter.

These studies describe the identification of a novel function for the amino acid transporter ATB^{0,+}. Since this transporter is expressed in the intestinal tract, lung, and mammary gland, it is likely to play a significant role in the handling of carnitine in these tissues.

A Na⁺-dependent transport system for carnitine has already been described. This transporter, known as OCTN2 (novel organic cation transporter 2), is expressed in most tissues and transports carnitine with high affinity. It is energized however only by a Na⁺ gradient and membrane potential. In contrast, ATB^{0,+} is a low-affinity transporter for carnitine, but exhibits much higher concentrative capacity than OCTN2 because of its energization by a Na⁺ gradient, a Cl⁻ gradient, and membrane potential.

In addition, the present inventors tested transport ability of ATB^{0,+} for D-amino acids. So far, no transporter on the intestinal lumen side that transports D-amino acids is known. The present inventors have revealed that ATB^{0,+} transports D-alanine, D-serine, D-methionine, D-leucine, and D-tryptophan. Moreover, the present inventors compared the transport ability of ATB^{0,+} for D-serine, whose importance in a living body was unraveled, with that of other transporters. As a result, it has been revealed that the transport ability of ATB^{0,+} for D-serine is higher than that of other many transporters.

In addition, the present inventors have discovered that phenylglycine is transported by ATB^{0,+} with higher affinity than other essential amino acids. Moreover, the present inventors also discovered a L-phenylglycine-derivative NOS inhibitor, such as (S)-(3-(2-nitroguanydyl)phenyl)glycine, is transported by ATB^{0,+} specifically.

In addition, the present inventors assessed the ability of aspartate- and glutamate-based prodrugs for the transport process mediated by mouse ATB^{0,+} in HRPE cells. As a result, it has been revealed that the methyl and benzyl esters of aspartate and glutamate are potent inhibitors of glycine transport. The present inventors also discovered L-glutamate γ-ester of acyclovir is transported by ATB^{0,+}. Furthermore, the present inventors demonstrated that even L-valine α-ester of acyclovir is transported by ATB^{0,+}.

Furthermore, the present inventors assessed the expression of ATB^{0,+} in the intestinal tract under inflammatory conditions. As a result, it has been revealed that inflammatory conditions lead to a marked increase in the expression of ATB^{0,+} in the ileum and colon. The present inventors also discovered up-regulation of ATB^{0,+} in tumor, such as breast tumor, and cancer cell lines, such as breast and hepatic cancers.

ATB^{0,+} can be an effective drug delivery system because, unlike other main amino acid transporters, it depends on three driving forces, Na⁺ gradient, Cl⁻ gradient, and membrane potential, therefore ATB^{0,+} is highly concentrative. In addition, because its tissue distribution or its expression is biased specifically to gastrointestinal tract (ileum and colon), lung, and mammary gland, it can be an effective delivery system for diseases specific to such tissues. Moreover, its expression is induced by pathology of, for example, enteritis, sepsis, and breast cancer; it can be an effective delivery system for the nidus of such pathology. Furthermore, it can be useful for delivery of prodrugs with amino acid structures because its substrate recognition is broad.

Therefore, an object of the present invention is to provide the use of amino acid transporter ATB^{0,+} as a delivery system for drugs and prodrugs. In a preferred embodiment of the present invention, drugs which are transported by ATB^{0,+} are NOS inhibitors, phenylglycine derivatives, carnitine, D-amino acids or derivatives thereof, or prodrugs with amino acid structures.

Another object of the present invention is to provide a method for screening of drugs which are suitable for being transported by ATB^{0,+} and to provide the drugs obtained by the screening.

A further object of the present invention is to provide a method for designing of drugs or prodrugs which are suitable for being transported by ATB^{0,+}. In a preferred embodiment of the present invention, the drugs have phenylglycine skeltone and the prodrugs have tyrosine, aspartic acid, glutamic acid, and 3- or 4-carboxyl phenylglycine as a moiety.

Thus, the present invention relates to use of amino acid transporter ATB^{0,+} as a delivery system for drugs and prodrugs. More specifically, the present invention provides:

(1) a method for screening for a drug or prodrug having ability to be transported by ATB^{0,+}, comprising the steps of:

(a) selecting compounds having the ability to be transported by ATB^{0,+};

(b) relating the selected compounds to a disease that can be treated and/or prevented with said compounds; and

(c) selecting a compound that is related to a disease in step (b);

(2) the method according to (1), wherein said compound having the ability to be transported by ATB^{0,+} is an NOS inhibitor, phenylglycine, carnitine or a D-amino acid, or a derivative thereof, and an amino acid-based prodrug;

(3) a method for designing a compound having the ability to be transported by ATB^{0,+}, wherein said method is provided for designing a compound selected from a group consisting of NOS inhibitors, phenylglycine, carnitine and D-amino acids, and derivatives thereof, and amino acid based prodrugs;

(4) a method for producing a compound having the ability to be transported by ATB^{0,+}, comprising the steps of:

(a) designing a compound selected from a group consisting of NOS inhibitors, phenylglycine, carnitine and D-amino acids, and derivatives thereof, and amino acid-based prodrugs; and

(b) synthesizing the designed compound;

(5) the method according to (4), further comprising the step of determining whether the synthesized compound has the ability to be transported by ATB^{0,+} to select a compound to be transported;

(6) a method for producing a drug containing, as an active ingredient, a compound with the ability to be transported by ATB^{0,+}, wherein said method comprises the steps of:

(a) designing a compound selected from a group consisting of NOS inhibitors, phenylglycine, carnitine and D-amino acids, and derivatives thereof, and amino acid-based prodrugs;

(b) synthesizing the designed compound; and

(c) determining whether the synthesized compound has the ability to be transported by ATB^{0,+} and selecting a compound to be transported;

(7) a method for producing a drug having, as an active ingredient, a compound with the ability to be transported by ATB^{0,+}, wherein said method comprises the steps of:

(a) designing a compound selected from a group consisting of NOS inhibitors, phenylglycine, carnitine and D-amino acids, and derivatives thereof, and amino acid-based prodrugs;

(b) synthesizing the designed compound; and

(c) relating the synthesized compound to a disease that can be treated and/or prevented with said compound;

(8) a method for transport of a compound mediated by ATB^{0,+}, wherein said compound is selected from a group consisting of NOS inhibitors, phenylglycine, carnitine and D-amino acids, and derivatives thereof, and amino acid-based prodrugs;

(9) a method for transport of a compound mediated by ATB^{0,+}, wherein said compound is selected from a group consisting of NOS inhibitors, phenylglycine, carnitine and D-amino acids, and derivatives thereof, and amino acid-based prodrugs, wherein said compound is labeled with a radioactive substance or conjugated with toxin;

(10) the method according to any one of (2) to (9), wherein said NOS inhibitor is a derivative of arginine, lysine, citrulline and ornithine;

(11) a therapeutic drug for a disease that can be treated and/or prevented with a compound selected from a group consisting of NOS inhibitors, phenylglycine, carnitine and D-amino acids, and derivatives thereof, wherein said therapeutic drug comprises the ATB^{0,+} gene as an active ingredient;

(12) a gene therapy for a disease that can be treated and/or prevented with a compound selected from a group consisting of NOS inhibitors, phenylglycine, carnitine and D-amino acids, and derivatives thereof, wherein said method comprises the step of administering the ATB^{0,+} gene;

(13) a therapeutic drug for cancer, comprising a compound having the ability to be transported by ATB^{0,+} as an active ingredient;

(14) the therapeutic drug according to (13), wherein said cancer is iNOS expressed cancer;

(15) the therapeutic drug according to (13), wherein said cancer is breast cancer or hepatic cancer;

(16) the therapeutic drug according to (13), wherein said compound is a NOS inhibitor;

(17) a method for treating cancer, comprising the step of administering a compound having the ability to be transported by ATB^{0,+};

(18) the method according to (17), wherein said cancer is iNOS expressed cancer;

(19) the method according to (17), wherein said cancer is breast cancer or hepatic cancer;

(20) the method according to (17), wherein said compound is a NOS inhibitor;

(21) use of a compound having the ability to be transported by ATB^{0,+} for producing a therapeutic drug for cancer;

(22) the use according to (21), wherein said cancer is iNOS expressed cancer;

(23) the use according to (21), wherein said cancer is breast cancer or hepatic cancer;

(24) the use according to (21), wherein said compound is a NOS inhibitor.

The present invention provides a method for screening for a drug or prodrug having ability to be transported by ATB^{0,+}. In the present invention, ATB^{0,+} is a Na⁺- and Cl⁻-coupled transport system for neutral and cationic amino acids. It plays an important role in the absorption of amino acids in the intestinal tract.

The compounds of the present invention include, but are not limited to, naturally occurring compounds, organic compounds, inorganic compounds, proteins, single compounds, such as peptides, and compound libraries, expression products from a gene library, cell extracts, supernatants from cell cultures, products from fermentation microorganisms, cell extracts from marine organisms, plant extracts, prokaryotic cell extracts, eukaryotic cell extracts, or animal cell extracts, NOS inhibitors, phenylglycine, carnitine or D-amino acids, or derivatives thereof, and amino acid-based prodrugs.

In the present invention, the NOS inhibitors are preferably the NOS inhibitors based on the structure of neutral and basic L-α-amino acids, more preferably, the NOS inhibitors based on the structure of L-arginine, L-lysin, L-citrulline, L-ornithine, even more preferably, the NOS inhibitors in Table 2 except for GGA.

In the present invention, L-phenylglycine derivatives are drugs and prodrugs with phenylglycine structure, more preferably, phenylglycine-derivative drug with free α-amino and α-carboxyl groups and no acidic group in the R group at physiological pH, or 3- or 4-carboxyl ester prodrug with 3- or 4-carboxyl phenylglycine. However, the parent drug should not be acidic at physiological pH.

In the present invention, carnitine is preferably carnitine and its derivatives, and carnitine and its acyl esters, and more preferably, carnitine, acetylcarnitine and propionylcarnitine.

In the present invention, D-amino acids are preferably D-alanine, D-serine, D-methionine, D-leucine, D-tryptophan, D-threonine, D-histidine, D-phenylalanine, D-glutamine and their derivatives, more preferably, D-alanine, D-serine, D-methionine, D-leucine, D-tryptophan and their derivatives, and even more preferably D-alanine, D-serine, D-methionine, D-leucine and D-tryptophan.

In the present invention, amino acid-based prodrugs preferably have an amino acid moiety (including D- and L-amino acids). For aspartate and glutamete, β-carboxyl ester prodrugs and γ-carboxyl ester prodrugs are preferred, respectively. For neutral and basic amino acids, α-carboxyl ester prodrugs are preferred.

In the present invention, the compounds described above may be adequately labeled and then used if necessary. Labels includes, for example, radioactive and fluorescent labels.

In the present invention, the methods for determining whether a compound is transported by ATB^{0,+} include, but are not limited to, those described in the working examples.

The screening method of the present invention subsequently investigates the relationship between the selected compound and the diseases that may be treated and/or prevented with the compound. The diseases to be tested for their relationship with the selected compound include, but are not limited to, those described below, if the selected compound is a NOS inhibitor: septic shock/hypotension (Titheradge Mass., Biochim Biophys Acta, 1999 1411(2-3): 437-55), anti-inflammatory, inflammatory bowel disease (Kubes P, McCafferty D M., Am J Med 2000; 109(2): 150-8), rheumatoid arthritis (Salerno L et.al., Curr Pharm Des 2002, 8(3): 177-200), glomerulonephritis (Cattell V., Kidney Int 2002 61(3):816-821), alzheimer's disease and stroke (Heneka M T and Feinstein D L, J Neuroimmunol 2001 114:8-18), cancer (Thomsen L L, Cancer Metastasis Rev 1998 17(1):107-18), HIV and HCV infection (Lake-Bakaar G et al., Dig Dis Sci 2001 46(5):1072-6), multiple sclerosis (Liu J S et al., Am J Pathol 2001 158(6):2057-66), asthma (Redington A E et al., Thorax 2001 56(5):351-7), osteoarthritis (Abramson S B et al., Curr Rheumatol Rep 2001 3(6):535-41), myocarditis (Gluck B et al., Herz 2000 25(3):255-60), glaucoma (Chiou G C, J Ocul Pharmacol Ther 2001 17(2):189-98), delayed hemorrhagic shock (Szabo C., New Horiz 1995 3(1):2-32), persistent pain (Meller S T et al., Neuropharmacology 1994, 33(11):1471-8), organ transplantation (Trajkovic V., Curr Drug Metab 2001, 2(3):315-29), and Parkinson's disease (Knott, C. et al., Mol Cell Neurosci 2000; 16(6):724-39). The following diseases may be tested if the selected compound is an L-phenylglycine derivative: the diseases described for NOS inhibitors, anti-influenza virus (Kati, W M. et al., Antimicrob Agents Chemother 2001, 45(9):2563-70), brain disorders and diseases such as ischemia and schizophreni (Pellicciari R and Costantino G, Curr Opin Chem Biol 1999 3(4):433-40; Conway S J et al., Bioorg Med Chem Lett. 2001, 11(6):777-80; Roberts P J., Neuropharmacology 1995, 34(8):813-9), and myocardinal ischaemia (Blackburn K J et al., Br. J. Pharmacol. 1979 66:443-444).

If the selected compound is carnitine, the following diseases may be tested: carnitine deficiencies including skeletal myopathy, cardiomyopathy, encephalopathy and failure to thrive (Kerner, J. and Hoppel, C., Annual Review of Nutrition, 18, 179-206, (1998); Treem, W. R. et al., New England Journal of Medicine, 319, 1331-1336, (1988).).

If the selected compound is a D-amino acid the following diseases may be tested: schizophrenia for D-serine and its derivatives (Heresco-Levy, U., Int. J. Neuropsychopharmacol.2000 3(3): 243-258., Krystal, J H. and D'Souza, D C., Biol. Psychiatry. 1998 44(11): 1075-76.).

If the selected compound is an amino acid-based prodrug, diseases related with a parent drug of the prodrug may be tested.

In the next step of the screening method of the present invention, a compound that has been related to a disease may be selected.

In the screening method of the present invention, the selected compound may be mixed with pharmaceutically acceptable carriers. A derivative of the selected compound may be synthesized and then mixed with pharmaceutically acceptable carriers.

The present invention provides a method for designing a compound having the ability to be transported by ATB^{0,+}, in which a compound is designed which is selected from a group consisting of NOS inhibitors, phenylglycine, carnitine and D-amino acids, and derivatives thereof, and amino acid-based prodrugs. Based on the fundamental structures of these compounds, novel compounds are designed in the method. For designing compounds, one skilled in the art may use any known methods.

The present invention also provides a method for producing a compound having the ability to be transported by ATB^{0,+}. In the method, initially, a compound is designed which is selected from a group consisting of NOS inhibitors, phenylglycine, carnitine and D-amino acids, and derivatives thereof, and amino acid-based prodrugs. In the next step, the designed compound is synthesized. The method further comprises the step of determining whether the synthesized compound has the ability to be transported by ATB^{0,+} to select a compound to be transported by ATB^{0,+}. Some of the compounds thus produced are thought to be pharmacologically active.

The present invention also provides a method for producing a drug containing, as an active ingredient, a compound with the ability to be transported by ATB^{0,+}. In the first step of the method, a compound is designed which is selected from a group consisting of NOS inhibitors, phenylglycine, carnitine and D-amino acids, and derivatives thereof, and amino acid-based prodrugs. In the second step, the designed compound is synthesized. In the third step, the synthesized compound may be related to a disease that can be treated and/or prevented with the compound, or the synthesized compound may be subjected to the determination whether it has the ability to be transported by ATB^{0,+} to be selected for the transport, followed by determination of the relationship between the synthesized compound and a disease that can be treated and/or prevented with the compound. In the method, a disease-related compound may further be selected and mixed with pharmaceutically acceptable carriers. A derivative of the selected compound may be synthesized and mixed with pharmaceutically acceptable carriers.

The present invention also-provides a method for transport of a compound mediated by ATB^{0,+}, in which the compound is selected from a group consisting of NOS inhibitors, carnitine and D-amino acids, and derivatives thereof, and amino acid-based prodrugs. In the method, the compound may be labeled. The labels include, but are not limited to, radioactive substances and toxins.

The present invention further provides for therapeutic use of the ATB^{0,+} gene for a disease that can be treated and/or prevented with a compound selected from a group consisting of NOS inhibitors, phenylglycine, carnitine and D-amino acids, and derivatives thereof. Such use includes, for example, therapeutic drugs containing the ATB^{0,+} gene as an active ingredient, which provides gene therapy for the diseases described above which comprises the step of administering the ATB^{0,+} gene. One skilled in the art would carry out the gene therapy, using a known method. The vectors used for the gene therapy of the present invention are not limited to any particular vectors.

The present invention also provides for use of a compound having the ability to be transported by ATB^{0,+}, e.g. NOS inhibitor, for treatment of cancer (preferably, iNOS expressed cancer, more preferably breast cancer and hepatic cancer). Such use includes, for example, a therapeutic for cancer comprising a compound having the ability to be transported by ATB^{0,+}, e.g. NOS inhibitor, as an active ingredient, cancer therapy comprising the step of administering a compound having the ability to be transported by ATB^{0,+}, e.g. NOS inhibitor, and use of a compound having the ability to be transported by ATB^{0,+}, e.g. NOS inhibitor, for production of therapeutics for cancer.

### BRIEF DESCRIPTION OF DRAWINGS

FIG. 1 shows a photograph indicating the result of Northern blot analysis of ATB^{0,+} mRNA along the longitudinal axis of the mouse intestinal tract. The small intestine was divided into four equal segments, the first segment representing the most proximal region and the fourth segment representing the most distal region of the small intestine. The blot was hybridized sequentially under high stringency conditions with [³²P]-labeled cDNA probes specific for mouse ATB^{0,+}, mouse PEPT1 and mouse β-actin.

FIG. 2 shows functional characteristics of mouse ATB^{0,+} in a mammalian cell expression system with glycine as the substrate. Results represent only ATB^{0,+}-specific transport activity which was calculated by subtracting the transport in vector-transfected cells from the transport in cDNA-transfected cells. A. Ion-dependence of ATB^{0,+}-mediated glycine (60 nM) transport. B. Saturation kinetics of ATB^{0,+}-mediated glycine transport. C. Na⁺-activation kinetics of ATB^{0,+}-mediated glycine (60 nM) transport. D. Cl⁻-activation kinetics of ATB^{0,+}-mediated glycine (60 nM) transport.

FIG. 3 shows the dose-response relationship for the inhibition of ATB^{0,+}-specific glycine (10 μM) transport by NOS inhibitors in HRPE cells expressing the cloned mouse ATB^{0,+}.

FIG. 4 shows characteristics of L-NNA transport via ATB^{0,+}. The cloned mouse AT^{0,+} was expressed in HRPE cells and the transport of [³H]-L-NNA (10 μM) was studied. Results represent only ATB^{0,+}-specific transport. A. Transport of L-NNA in vector-transfected cells and in ATB^{0,+} cDNA-transfected cells. B. Inhibition of ATB^{0,+}-specific L-NNA transport by amino acids (5 mM). C. Saturation kinetics of L-NNA transport via ATB^{0,+}.

FIG. 5 shows ion-dependence of inward currents induced by L-arginine, L-NIL, and L-MTC in X. laevis oocytes expressing the cloned mouse ATB^{0,+}. Oocytes were perifused with 1 mM substrates in buffers containing NaCl, sodium gluconate, or NMDG chloride.

FIG. 6 shows saturation kinetics for NOS inhibitors for transport via ATB^{0,+} in X. laevis oocyte expression system. Oocytes expressing the cloned mouse ATB^{0,+} were perifused with increasing concentrations of L-MTC, L-NIL, and L-NIO and the inward currents were measured under voltage clamp conditions. Upper panels represent the relationship between substrate concentration and inward current at −50 mV. Lower panels represent the relationship between K_{0.5} values and membrane potential.

FIG. 7 shows inhibition of mouse ATB^{0,+}-mediated glycine transport by carnitine and its acyl esters in HRPE cells. A, transport of glycine (10 μM) in vector-transfected cells and in cells transfected with mouse ATB^{0,+} cDNA(mATB^{0,+}). B, inhibition of ATB^{0,+}-mediated glycine (10 μM) transport by γ-aminobutyrate (GABA), betaine, carnitine, acetylcarnitine, and propionylcarnitine. Transport in the absence of inhibitors was taken as 100%.

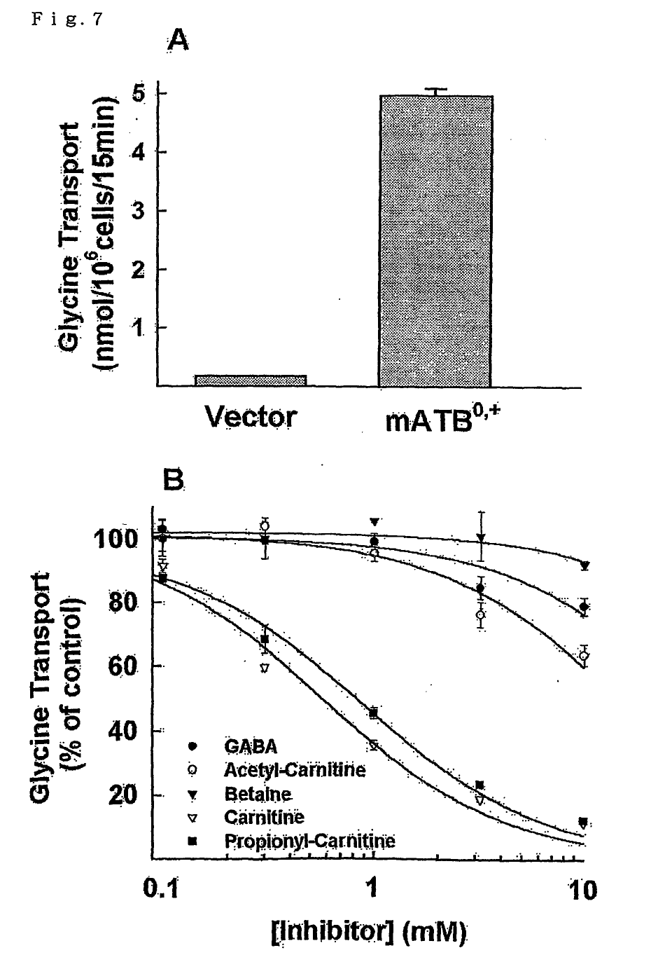

FIG. 8 shows transport of carnitine and its acyl esters by mouse ATB^{0,+} and the ion-dependence of the process in HRPE cells. A, transport of carnitine (25 μM), propionylcarnitine (25 μM), and acetylcarnitine (25 μM) in vector-transfected cells and in cells transfected with mouse ATB^{0,+} cDNA. B, transport of carnitine (15 μM) in vector-transfected cells and in cells transfected with mouse ATB^{0,+} cDNA in the presence of NaCl, in the presence of Na⁺ but in the absence of Cl⁻ (Na gluconate), and in the presence of Cl⁻ but in the absence of Na⁺ (NMDG-Cl).

FIG. 9 shows saturation kinetics and Na⁺-activation kinetics of mouse ATB^{0,+}-mediated carnitine transport HRPE cells. A, transport of carnitine via mouse ATB^{0,+} over a carnitine concentration range of 0.1-5 mM (inset, Eadie-Hofstee plot; V, carnitine transport in nmol/10⁶ cells/15 min; S, carnitine concentration in mM). B, transport of carnitine (15 μM) via mouse ATB^{0,+} over a Na⁺ concentration range of 2.5-140 mM.

FIG. 10 shows characteristics of carnitine transport mediated by mouse ATB^{0,+} in Xenopus laevis oocytes. A, saturation kinetics for carnitine-induced current in oocytes expressing mouse ATB^{0,+} (inset, Eadie-Hofstee plot; I, carnitine-induced current; S, carnitine concentration in mM). B, Na⁺-dependence of carnitine (1 mM)-induced current in oocytes expressing mouse ATB^{0,+} (inset, Hill plot; I, carnitine-induced current; Iₘₐₓ, maximal current induced by 1 mM carnitine). C, Cl⁻-dependence of carnitine (1 mM)-induced current in oocytes expressing mouse ATB^{0,+} (inset, Hill plot; I, carnitine-induced current; Iₘₐₓ, maximal current induced by 1 mM carnitine).

FIG. 11 shows D-serine transport by 10 different amino acid transporters which are expressed in the intestinal tract. Transport of D-[³H]-serine (5 μM) was measured in HRPE cells transfected with cDNA of each transporter.

FIG. 12 shows dose-response relationship for the inhibition of mouse ATB^{0,+}-mediated [³H]glycine (57 nM) uptake.

FIG. 13 shows transport of L-phenylglycine in vector-transfected cells and mouse ATB^{0,+}, human ATB⁰, mouse b^{0,+}AT/rBAT cDNA-transfected cells. The cloned transporters were expressed in HPRE cells and the transport of [¹⁴C]phenylglycine (10 μM) was studied with or without Na⁺.

FIG. 14 shows the process of the (S)-(3-(2-nitroguanydyl)phenyl)glycine synthesis.

FIG. 15 shows dose-response relationship of inward currents induced by (S)-(3-(2-nitroguanydyl)phenyl)glycine (A) and L-NNA (B) in X. laevis oocytes expressing the cloned mouse ATB^{0,+}.

FIG. 16 shows Na⁺- and Cl⁻-coupled active transport of aspartate- and glutamate-based prodrugs by the amino acid transporter ATB^{0,+}. Mouse ATB^{0,+} cDNA-specific transport of glycine (10 μM) was measured in HRPE cells following heterologous expression of the cDNA. The concentration of aspartate derivatives (A) and glutamate derivatives (B) was varied as indicated. Results are given as % of control transport measured in the absence of inhibitors.

FIG. 17 shows dose-response relationship of inward currents induced by L-glutamate γ-ester of acyclovir in X. laevis oocytes expressing the cloned mouse ATB^{0,+}.

FIG. 18 shows transport of [8-³H]-valacyclovir in vector-transfected cells and mouse ATB^{0,+} cDNA-transfected cells. The cloned transporters were expressed in HPRE cells and the transport of [8-³H]-valacyclovir (1.25 μM) was studied.

FIG. 19 shows photographs indicating up-regulation of ATB^{0,+} expression in the intestinal tract under inflammatory conditions. Mice were injected i.p. with either saline (control) or bacterial lipopolysaccharide (5 mg/kg body wt). 16 h following injection, mice were killed and the various segments of the intestine (J, jejunum; L, ileum; C, colon) were collected for mRNA isolation. Five-week-old IL2^{+/+} and IL2^{-/-} mice were killed and the various segments of the intestine were collected for mRNA isolation. Semiquantitative RT-PCR with low number of PCR cycles was carried out with primer pairs specific for mouse ATB^{0,+}, mouse glyceraldehyde-3-phosphate dehydrogenase (GAPDH), and mouse cyclophilin C (CypC) and the RT-PCR products were detected by Southern hybridization and the signals were quantified.

FIG. 20 shows a photograph indicating up-regulation of ATB^{0,+} and iNOS expression in the mouse breast tumor.

### BEST MODE FOR CARRYING OUT THE INVENTION

The following examples illustrate the present invention in more detail, but are not to be construed to limit the scope of the present invention.

1. Na⁺- and Cl⁻-Coupled Active Transport of Nitric Oxide Synthase Inhibitors via the Amino Acid Transporter ATB^{0,+}

Methods

Materials.

[³H]-Glycine was purchased from Moravek (Brea, Calif.) and [³H]-L-NNA was purchased from Amersham Pharmacia Biotech(Piscataway, N.J.). All other radiolabeled amino acids were obtained from either Dupont-New England Nuclear (Boston, Mass.) or American Radiolabeled Chemicals, Inc. (St. Louis, Mo.). NOS inhibitors were obtained from either Sigma or Calbiochem.

Cloning of Mouse ATB^{0,+}.

The SuperScript plasmid system (Life Technologies, Inc.) was used to establish a unidirectional cDNA library with poly(A)⁺ RNA isolated from mouse colon as described previously (Sugawara, M. et al., J.Biol.Chem. 275, 16473-16477, (2000), Fei, Y. J. et al., J.Biol.Chem., 275, 23707-23717, (2000), Wang, H. et al., Am.J.Physiol., 278, C1019-C1030, (2000)). The probe for library screening was prepared by RT-PCR using primers specific for mouse ATB^{0,+} cDNA reported in the GenBank (accession no. AF161714). The primers were 5′-GTT GGC TAT GCA GTG GGA TT-3′ (sense) and 5′-GAG GCC AAG GAG AAA CAA AA-3′ (antisense) which corresponded to the nucleotide positions 396-415 and 1606-1625 in the cDNA sequence. RT-PCR was done using the poly(A)⁺ RNA prepared from mouse colon and the resulting product, ˜1.2 kbp in size, was subcloned and sequenced to confirm its identity. This cDNA was labeled with [α-³²P]dCTP by random priming and used as a probe for screening the mouse colon cDNA library. Sequencing was done using an automated Perkin-Elmer Applied Biosystems 377 Prism DNA sequencer. The longest positive clone (˜3 kbp) was used for functional studies.

Northern Blot.

The expression pattern of ATB^{0,+} mRNA along the longitudinal axis of the mouse intestinal tract was investigated by Northern blot analysis. The entire small intestine was divided into four equal segments, the first segment representing the most proximal small intestine and the fourth segment representing the most distal small intestine. Poly(A)⁺ mRNA was isolated from these four segments as well as from the cecum and colon and used for Northern blot analysis. The blot was hybridized sequentially under high stringency conditions with [³²P]-labeled cDNA probes specific for mouse ATB^{0,+}, mouse PEPT1 and mouse β-actin.

Functional Expression ATB^{0,+} in HRPE Cells.

This was done using the vaccinia virus expression system (Sugawara, M. et al., J.Biol.Chem. 275, 16473-16477, (2000), Fei, Y. J. et al., J.Biol.Chem., 275, 23707-23717, (2000), Wang, H. et al., Am.J.Physiol., 278, C1019-C1030, (2000)). Transport measurements were made at 37° C. for 15 min with radiolabeled amino acids or NOS inhibitors as substrates. The transport buffer was 25 mM Hepes/Tris (pH 7.5) containing 140 mM NaCl, 5.4 mM KCl, 1.8 mM CaCl₂, 0.8 mM MgSO₄, and 5 mM glucose. Endogenous transport activity was always determined in parallel using cells transfected with vector alone. With glycine as the substrate which was used in most of the experiments, the endogenous transport accounted for <5% of the transport measured in cells that were transfected with the cDNA. cDNA-specific transport was calculated by adjusting for the endogenous activity. The kinetic parameters, Michaelis-Menten constant (Kₜ) and maximal velocity (Vₘₐₓ), were calculated by fitting the cDNA-specific transport data to the Michaelis-Menten equation describing a single saturable transport system. The Na⁺- and Cl⁻-activation kinetics were analyzed by fitting the cDNA-specific transport data to the Hill equation, and the Hill coefficient was calculated.

Functional Expression ATB^{0,+} in X. laevis oocytes.

Capped cRNA from the cloned mouse ATB^{0,+} cDNA was synthesized using the mMESSAGE mMACHINE™ kit (Ambion). Mature oocytes from X. laevis were isolated by treatment with collagenase A (1.6 mg/ml), manually defolliculated, and maintained at 18° C. in modified Barth's medium supplemented with 10 mg/ml gentamycin (Sugawara, M. et al., J.Biol.Chem. 275, 16473-16477, (2000), Fei, Y. J. et al., J.Biol.Chem., 275, 23707-23717, (2000), Wang, H. et al., Am.J.Physiol., 278, C1019-C1030, (2000)). On the following day, oocytes were injected with 50 ng cRNA. Uninjected oocytes served as controls. The oocytes were used for electrophysiological studies 6 days after cRNA injection. Electrophysiological studies were done by the two-microelectrode voltage-clamp method (Sugawara, M. et al., J.Biol.Chem. 275,16473-16477, (2000), Fei, Y. J. et al., J.Biol.Chem., 275, 23707-23717, (2000), Wang, H. et al., Am.J.Physiol., 278, C1019-C1030, (2000)). Oocytes were perifused with a NaCl-containing buffer (100 mM NaCl, 2 mM KCl, 1 mM MgCl₂, 1 mM CaCl₂, 3 mM Hepes, 3 mM Mes, and 3 mM Tris, pH 7.5), followed by the same buffer containing different NOS inhibitors or amino acids. The membrane potential was clamped at −50 mV. Voltage pulses between +50 and −150 mV, in 20-mV increments, were applied for 100-ms durations and steady-state currents were measured. The differences between the steady-state currents measured in the presence and absence of substrates were considered as the substrate-induced currents. The kinetic parameter K_{0.5} (i.e., the substrate concentration necessary for the induction of half-maximal current) for the saturable transport of substrates was calculated by fitting the values of the substrate-induced currents to the Michaelis-Menten equation. The experiments were repeated with at least three different oocytes from two different batches.

Results

Structural Features of Mouse ATB^{0,+}.

The cloned mouse ATB^{0,+} cDNA obtained from colon mRNA is 3,007 bp-long (GenBank accession no. AF320226) and codes for a protein of 638 amino acids. The primary structure of mouse ATB^{0,+} is highly homologous to the recently cloned human ATB^{0,+} (Sloan, J. L. and Mager, S. J.Biol.Chem., 274, 23740-23745, (1999)). The identity of amino acid sequence between the two proteins is 88%.

Expression Pattern of ATB^{0,+} mRNA in the Mouse Intestinal Tract.

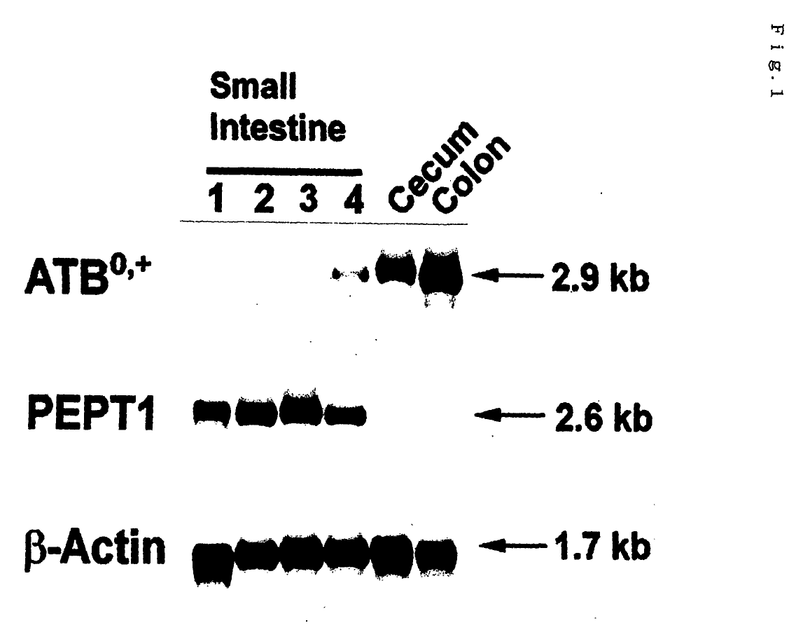

To determine the expression pattern of ATB^{0,+} along the longitudinal axis of the intestinal tract, we analyzed the steady-state levels of ATB^{0,+} mRNA in different regions of the mouse intestine by Northern blot hybridization (FIG. 1). ATB^{0,+} mRNA was not detectable in the first three segments of the small intestine. The expression of the mRNA was however evident in the fourth segment of the small intestine, cecum, and colon. The mRNA levels were more abundant in the colon and cecum than in the distal small intestine. In contrast, mRNA for PEPT1, a H⁺-coupled transporter for small peptides, was detectable in all four segments of the small intestine but not in the cecum and colon. These data show that the expression of ATB^{0,+} mRNA is restricted to the distal region of the mouse intestinal tract.

Functional Features of Mouse ATB^{0,+}.

The functional identity of the cloned mouse ATB^{0,+} cDNA was established first by expressing the clone in mammalian cells heterologously and studying its transport function. We studied the transport of several zwitterionic and cationic amino acids in HRPE cells expressing the cloned mouse ATB^{0,+}. The transport of these amino acids was also measured under identical conditions in cells transfected with vector alone to serve as a control for endogenous transport activity. The transport activity of all 13 amino acids tested (Gly, Ala, Ser, Thr, Pro, His, Gln, Asn, Leu, Ile, Phe, Trp, and Arg) was found to be significantly higher in mouse ATB^{0,+} cDNA-transfested cells than in vector-transfected cells (data not shown). The cDNA-induced increase in transport activity varied between 16-190% for all amino acids, except for glycine. The transport of glycine was exceptionally high in cDNA-transfected cells. The increase was 38-fold compared to transport activity in vector-transfected cells.

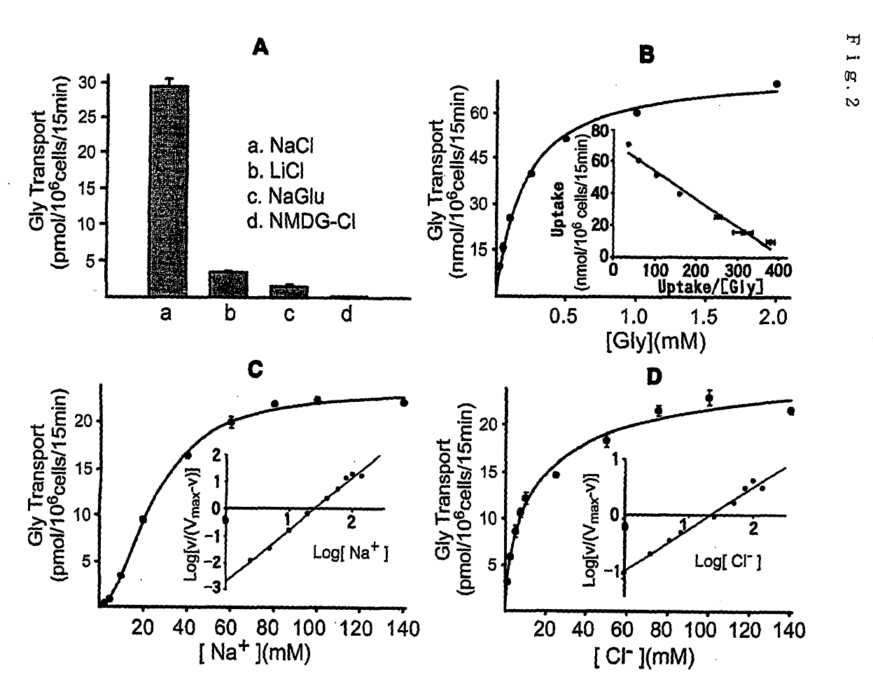

Since the transport of glycine via mouse ATB^{0,+} was markedly higher compared to the transport of other amino acids, we used glycine as the substrate for further characterization of the cloned transporter. The results given in FIG. 2 represent only the ATB^{0,+}-specific transport activity after correcting for the endogenous transport activity. The ATB^{0,+}-mediated glycine transport was obligatorily dependent on the presence of Na⁺ and Cl⁻. The transport of glycine via mouse ATB^{0,+} was saturable with a Michaelis-Menten constant (Kₜ) of 210±18 μM. The number of Na⁺ and Cl⁻ ions involved in the transport process was then analyzed by the Na⁺-activation kinetics and the Cl⁻-activation kinetics. The transport activity of mouse ATB^{0,+} was sigmoidally related to the concentration of Na⁺ and the Hill coefficient for the activation process for Na⁺ was 2.0±0.1. In contrast, the transport activity of mouse ATB^{0,+} showed a hyperbolic relationship with the concentration of Cl⁻. The Hill coefficient for the activation process for Cl⁻ was 0.7-0.1. These results show that the Na⁺: Cl⁻: glycine stoichiometry is 2:1:1. The K_{0.5} values for Na⁺ and Cl⁻ (i.e., the concentrations of these ions needed for inducing half-maximal transport activity) were 25±1 and 18±8 mM, respectively.

The amino acid specificity of mouse ATB^{0,+} was then studied by assessing the ability of a variety of amino acids to compete with [³H]-glycine (60 nM) for the transport process mediated by the cloned transporter (Table 1).

TABLE 1
Amino Acid Substrate Specificity of Mouse ATB^{0,+}

| | mATB^{0,+}-Specific | |
|---|---|---|
| Unlabeled | [³H]-Glycine Transport | |
| Amino Acid | pmol/10⁶ cells/15 min | % |
| Control | 37.27 ± 1.20 | 100 |
| Glycine | 1.88 ± 0.06 | 5 |
| Alanine | 1.04 ± 0.05 | 3 |
| Cysteine | 1.11 ± 0.20 | 3 |
| Serine | 1.99 ± 0.09 | 5 |
| Threonine | 3.91 ± 0.30 | 11 |
| Proline | 9.97 ± 0.57 | 27 |
| Histidine | 1.31 ± 0.10 | 4 |
| Glutamine | 2.39 ± 0.18 | 6 |
| Asparagine | 3.25 ± 0.23 | 9 |
| Leucine | 0.27 ± 0.03 | 1 |
| Isoleucine | 0.27 ± 0.02 | 1 |
| Phenylalanine | 0.56 ± 0.03 | 2 |
| Tryptophan | 0.37 ± 0.06 | 1 |
| Arginine | 7.14 ± 0.35 | 19 |
| Lysine | 3.52 ± 0.28 | 9 |
| Aspartate | 32.01 ± 1.29 | 86 |
| Glutamate | 33.47 ± 0.91 | 90 |
| MeAIB | 36.21 ± 4.80 | 97 |

Transport of [³H]-glycine (60 nM) was measured in vector-transfected HRPE cells and in ATB^{0,+} cDNA-transfected HRPE cells at 37° C. for 15 min in the presence of NaCl (pH 7.5). Unlabeled amino acids were used at a concentration of 2.5 mM. cDNA-specific transport was calculated by subtracting the transport in vector-transfected cells from the transport in mATB^{0,+} cDNA-transfected cells. Data (means±SEM from four separate determinations) represent only cDNA-specific transport.

At a concentration of 2.5 mM, all zwitterionic and cationic amino acids tested inhibited the transport of [³H]-glycine mediated by mouse ATB^{0,+}. The inhibition varied from 70 to 100%. In contrast, the anionic amino acid aspartate and the zwitterionic N-methylated amino acid, MeAIB, did not show any significant inhibition. These data show that the cloned mouse ATB^{0,+} is capable of mediating the transport ic and cationic amino acids in a Na⁺- and Cl⁻-coupled manner.

Transport of NOS Inhibitors via ATB^{0,+}.

The present inventors then assessed the ability of NOS inhibitors and their parent amino acids (arginine, lysine, ornithine, and citrulline) at a concentration of 2.5 mM to compete with [³H]-glycine (10 μM) for the transport process mediated by mouse ATB^{0,+} in HRPE cells (Table 2).

TABLE 2
Transport of NOS Inhibitors via Mouse ATB^{0,+}

| | mATB^{0,+}-Specific | |
|---|---|---|
| Unlabeled | [³H]-Glycine Transport | Current in |
| Amino Acid or | in HRPE cells | X. laevis oocytes |
| NOS Inhibitor | nmol/10⁶ cells/15 min | nA |
| Control | 7.29 ± 0.08 (100) | |
| Arginine | 2.21 ± 0.05 (30) | 382 ± 57 |
| Lysine | 1.48 ± 0.02 (20) | 545 ± 95 |
| Citrulline | 1.10 ± 0.01 (15) | 498 ± 114 |
| Ornithine | 3.28 ± 0.01 (45) | 131 ± 27 |
| L-NNA | 1.54 ± 0.03 (21) | 200 ± 92 |
| L-NAME | 5.25 ± 0.10 (72) | 11 ± 2 |
| L-NMMHA | 5.21 ± 0.06 (72) | 33 ± 6 |
| L-NDMA | 4.25 ± 0.10 (58) | 85 ± 15 |
| L-NMEA | 3.77 ± 0.17 (52) | 138 ± 16 |
| L-NMMA | 2.60 ± 0.06 (36) | 411 ± 76 |
| L-NABE | 4.93 ± 0.19 (67) | 16 ± 2 |
| L-NIL | 4.77 ± 0.73 (65) | 89 ± 18 |
| L-TC | 1.05 ± 0.06 (15) | 497 ± 104 |
| L-MTC | 2.03 ± 0.09 (28) | 118 ± 34 |
| L-NIO | 4.17 ± 0.10 (57) | 71 ± 16 |
| L-Canavanine | 1.75 ± 0.03 (24) | 269 ± 73 |
| GGA | 4.22 ± 0.09 (58) | 7 ± 1 |

Transport of [³H]-glycine (10 μM) was measured in vector-transfected HRPE cells and in mATB^{0,+} cDNA-transfected HRPE cells at 37° C. for 15 min in the presence of NaCl (pH 7.5). Unlabeled amino acids and NOS inhibitors were used at a concentration of 2.5 mM. cDNA-specific transport was calculated by subtracting the transport in vector-transfected cells from the transport in mATB^{0,+} cDNA-transfected cells. Values in parenthesis are percent of control transport. Data (means±SEM from four separate determinations) represent only cDNA-specific transport. mATB^{0,+} was also expressed in X. laevis oocytes by injecting mATB^{0,+} cRNA, and the inward currents induced by amino acids and NOS inhibitors (1 mM) were measured using the two-microelectrode voltage clamp technique. The perifusion medium contained NaCl (pH 7.5). Data represent means±SEM from three different batches of oocytes.

All four parent amino acids caused marked inhibition of glycine transport via mouse ATB^{0,+}. The inhibition varied between 55-85%. Similarly, all NOS inhibitors that were tested also inhibited ATB^{0,+}-mediated glycine transport. The inhibition caused by the arginine-based NOS inhibitors L-NNA, L-NAME, L-NMMHA, L-NDMA, L-NMEA, L-NMMA, and L-NABE was in the range of 30-80%. The lysine-based NOS inhibitor L-NIL caused 35% inhibition. L-TC and L-MTC, the citrulline-based NOS inhibitors, were very potent as inhibitors of ATB^{0,+}-mediated glycine transport, the inhibition being in the range of 70-85%. The ornithine derivative L-NIO caused 40% inhibition. L-Canavanine and L-GGA, both being guanidino derivatives, were also effective inhibitors, causing 75 and 40% inhibition, respectively.

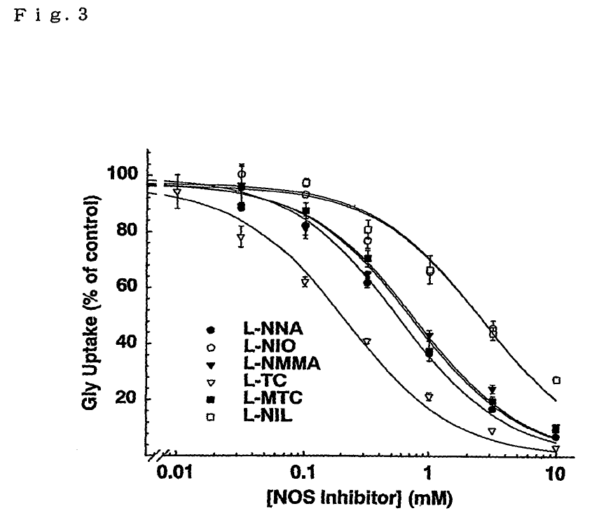

FIG. 3 describes the dose-response relationship for the inhibition of ATB^{0,+}-mediated glycine transport by six of the NOS inhibitors. The inhibitory potency was in the following order: L-TC>L-NNA>L-MTC=L-NMMA>L-NIO=L-NIL. The IC₅₀ values (i.e., the concentration of the compound necessary to cause 50% inhibition) were as follows: L-TC (0.21±0.03 mM), L-NNA (0.56±0.04 mM), L-MTC (0.73±0.09 mM), L-NMMA (0.77±0.12 mM), L-NIO (2.65±0.25 mM), and L-NIL (2.60±0.29 mM).

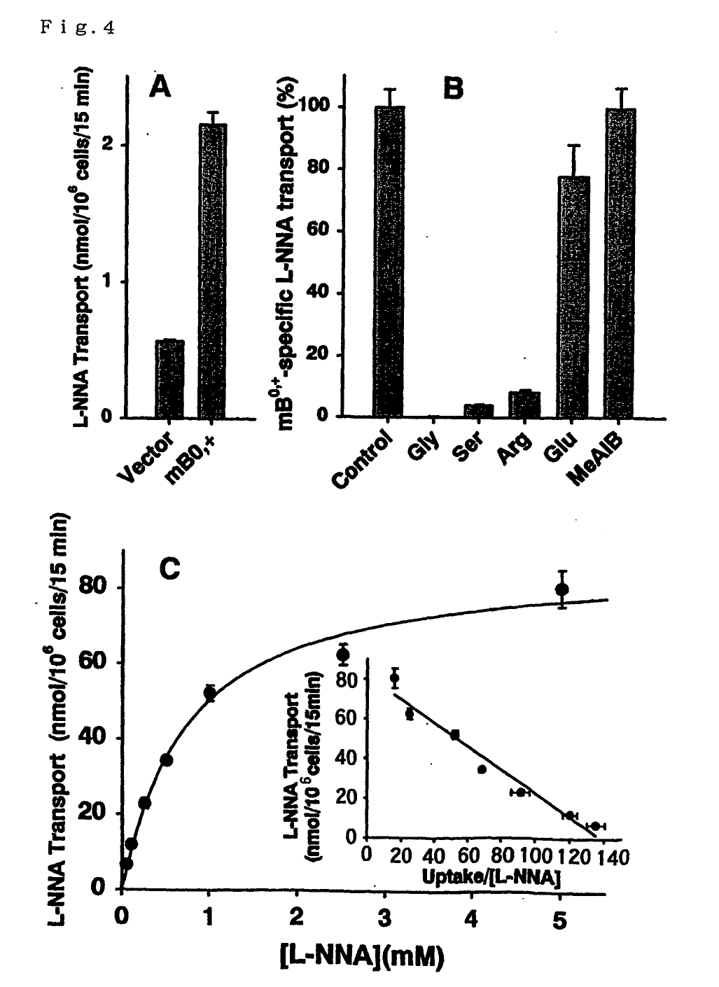

These results show that all NOS inhibitors tested interact with the substrate-binding site of the cloned mouse ATB^{0,+}. However, these data suggest but do not prove that these NOS inhibitors are transportable substrates for the transporter. It is possible that some compounds may block the transport by competing with glycine for binding to the substrate-binding site without itself being transported across the membrane. To determine whether or not these inhibitors are actually transportable substrates of the transporter, direct measurements of ATB^{0,+}-mediated transport of these inhibitors have to be carried out. Towards this goal, we used [³H]-L-NNA as a substrate for ATB^{0,+} and studied its transport in HRPE cells expressing the cloned transporter (FIG. 4). The transport of L-NNA in ATB^{0,+}-expressing cells was 4-fold higher than in vector-transfected cells, demonstrating that L-NNA is indeed a transportable substrate for this transporter. This conclusion is supported further by the inhibition of ATB^{0,+}-specific L-NNA transport by glycine, serine and arginine that are substrates for the transporter. In contrast, MeAIB and glutamate that are not substrates for the transporter did not inhibit ATB^{0,+}-specific L-NNA transport. The ATB^{0,+}-specific L-NNA transport was saturable with a Michaelis-Menten constant of 0.75±0.10 mM.

The mammalian expression system is not ideal to investigate the transport of a broad spectrum of NOS inhibitors via ATB^{0,+} because of the limited availability of NOS inhibitors commercially in radiolabeled form. Therefore, the present inventors had to use an alternative method for direct measurement of the transport of a large number of NOS inhibitors via ATB^{0,+}. The present inventors used the X. laevis oocyte expression system for this purpose. The cloned mouse ATB^{0,+} was functionally expressed in these oocytes by injection of cRNA and the transport of NOS inhibitors (1 mM) via the transporter was then monitored by inward currents induced by these inhibitors using the two-microelectrode voltage clamp technique. This approach was feasible because of the electrogenic nature of ATB^{0,+}. Induction of an inward current upon exposure of the ATB^{0,+}-expressing oocyte to a test compound under voltage-clamped conditions would indicate depolarization of the membrane as a result of transport of the compound into the oocyte. Uninjected oocytes served as negative controls in these experiments. The results of these oocyte experiments are given in Table II. All of the NOS inhibitors tested, except for L-NAME, L-NABE, and L-GGA, induced marked inward currents in oocytes expressing the cloned ATB^{0,+}. The currents varied in the range of 30-500 nA. Comparatively, L-NAME, L-NABE, and L-GGA induced very little currents (10-15 nA). The amino acids arginine, lysine, citrulline, and ornithine induced currents in the range of 130-550 nA.

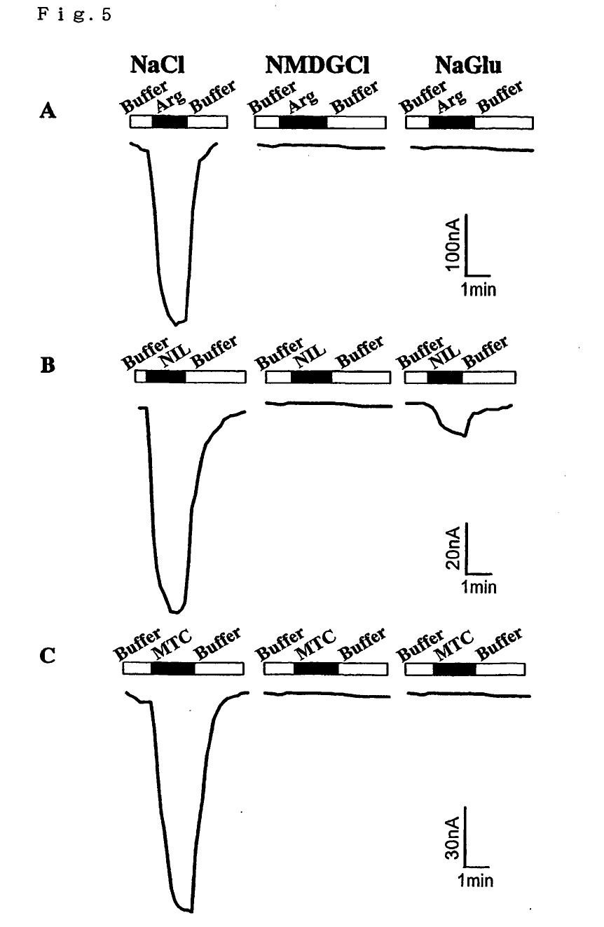

For further detailed analysis of the transport of NOS inhibitors via ATB^{0,+} in the oocyte expression system, the present inventors selected three NOS inhibitors, namely L-NIL, L-MTC, and L-NIO. The present inventors chose these compounds on the basis of their selectivity towards distinct NOS isoforms: L-NIL for NOS II, L-MTC for NOS I and L-NIO for NOS III. FIG. 5 describes the ion-dependence of the inward currents induced by L-NIL and L-MTC. In the presence of NaCl, L-NIL at a concentration of 1 mM induced 82±18 nA inward currents. However, when measured in the presence of NMDG chloride, there was no measurable inward current upon exposure of the oocytes to this compound, indicating that the L-NIL-induced currents were obligatorily dependent on the presence of Na⁺. This compound did induce a small, but significant, current (˜10 nA) in the presence of sodium gluconate (i.e., in the absence of chloride). However, since L-NIL used in this experiment was a chloride salt, a small amount of chloride was present under these experimental conditions. This resulted in the induction of the observed current. This is supported by the data obtained with L-MTC which is available in a chloride-free form. In the presence of NaCl, L-MTC at a concentration of 1 mM induced 116±34 nA inward currents. But, there were no measurable currents when either Na⁺ or Cl⁻ was absent, showing that the L-MTC-induced inward current was absolutely dependent on the presence of both Na⁺ and Cl⁻. The obligatory dependence of the currents induced by L-NIL and L-MTC on the presence of Na⁺ and Cl⁻ was similar to the data obtained with arginine. When used as a chloride-free salt, arginine induced 382±57 nA inward current. No measurable current was observed with arginine in the absence of either Na⁺ or Cl⁻.

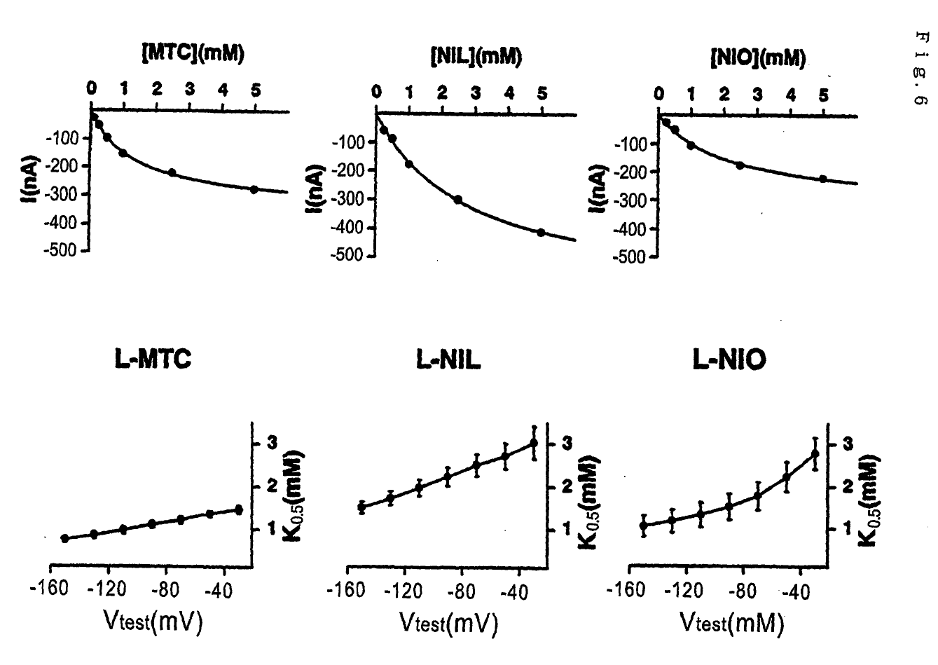

The present inventors then analyzed the saturation kinetics of the transport of L-MTC, L-NIL, and L-NIO via ATB^{0,+} in the oocyte expression system using the inward currents induced by the corresponding inhibitors as the measure of their transport. The results of these experiments, shown in FIG. 6, demonstrate that ATB^{0,+}-mediated transport of all three compounds was saturable. The K_{0.5} values (the concentration of the compound necessary to induce half-maximal current), calculated at membrane potential −50 mV, were 1.36±0.08 mM, 2.72±0.29 mM, and 2.24±0.35 mM for L-MTC, L-NIL, and L-NIO, respectively. The K_{0.5} values were however influenced by the membrane potential for all three compounds. The values decreased with hyperpolarization of the membrane and increased with depolarization of the membrane.

Discussion

The present inventors present evidence for the transport of NOS inhibitors via the Na⁺- and Cl⁻-coupled amino acid transporter ATB^{0,+}. The present invention represents the first identification of an ion gradient-driven concentrative transport system for these potential therapeutic agents. The evidence in support of ATB^{0,+}-mediated transport of NOS inhibitors was obtained with ATB^{0,+} cloned from the mouse colon. The transport function of the cloned ATB^{0,+} was studied by heterologous expression in mammalian cells as well as in X. laevis oocytes. This approach enabled us to investigate directly the transport of a wide variety of NOS inhibitors via the transporter. NOS inhibitors can be either cationic or zwitterionic in nature. Previous studies have shown that cationic NOS inhibitors are transported by system y⁺ whereas zwitterionic NOS inhibitors are transported by system L (Schmidt, K. et al., Mol.Pharmacol., 44, 615-621, (1993), Schmidt, K et al., Biochem.J., 301, 313-316, (1994), Baydoun, A. R. and Mann, G. E., Biochem.Biophys.Res.Commun., 200, 726-731, (1994), Schmidt, K. et al., J.Neurochem., 64, 1469-1475, (1995), Raghavendra Rao, V. L. and Butterworth, R. F., J.Neurochem., 67, 1275-1281, (1996)). System y⁺ does not interact with zwitterionic NOS inhibitors and system L does not interact with cationic NOS inhibitors. Our present studies show that ATB^{0,+} is able to transport both cationic as well as zwitterionic NOS inhibitors. This is in accordance with the substrate specificity of this transporter. ATB^{0,+} recognizes cationic amino acids as well as zwitterionic amino acids as substrates. Among the amino acid substrates of ATB^{0,+}, how the amino acids arginine, lysine, citrulline, and ornithine are handled by the transporter is directly relevant to the present study because most of the NOS inhibitors that are currently in investigational use are structurally related to these four amino acids. Even though arginine, lysine, and ornithine are cationic and citrulline is zwitterionic, our present studies show that all of these four amino acids are transportable substrates for ATB^{0,+}. Similarly, most NOS inhibitors, whether cationic or zwitterionic, that are structurally related to the four amino acids are transported via ATB^{0,+}.

Among the three amino acid transporters that are known thus far to recognize NOS inhibitors as substrates, ATB^{0,+} is the most concentrative. This transporter is energized by the combined transmembrane gradients of Na⁺ and Cl⁻ as well as membrane potential. In contrast, system y⁺ is driven only by membrane potential and system L is most likely facilitative with no known driving force. Therefore, the transport of NOS inhibitors into cells that express ATB^{0,+} is likely to be highly concentrative. The intracellular concentration of the NOS inhibitors in these cells can potentially reach several-fold higher than the extracellular concentration.

The present inventors determined the affinity of six NOS inhibitors for ATB^{0,+} in the mammalian cell expression system from their ability to compete with an amino acid substrate for the transport process. These are L-NNA and L-NMMA (both of these structurally related to arginine), L-NIL (structurally related to lysine), L-TC and L-MTC (both of these structurally related to citrulline) and L-NIO (structurally related to ornithine). The IC₅₀ values for these six compounds varied within the range of 0.2-2.7 mM. In the case of L-NNA, the present inventors also determined the affinity directly from its transport via ATB^{0,+} in the same mammalian cell expression system. The Michaelis-Menten constant (Kₜ) calculated from the direct measurement of transport was found to be very similar to the IC₅₀ value calculated from the competitive inhibition studies (0.75±0.10 mM versus 0.56±0.04 mM). For three other NOS inhibitors (L-MTC, L-NIL, and L-NIO), the present inventors determined the Michaelis-Menten constant for their transport via ATB^{0,+} using the X. laevis oocyte expression system. The Kₜ values calculated from these experiments were found to be similar to the corresponding IC₅₀ values determined from the competitive inhibition studies using the mammalian cell expression system (1.36±0.08 mM versus 0.73±0.09 mM for L-MTC, 2.72±0.29 mM versus 2.60±0.29 mM for L-NIL, and 2.24±0.35 mM versus 2.65±0.25 mM for L-NIO).

The transport of NOS inhibitors via ATB^{0,+} is of significant pharmacological and clinical relevance. This suggests that ATB^{0,+} has the potential for use as a drug delivery system for NOS inhibitors. The present inventors cloned ATB^{0,+} from the mouse colon. But, there is ample evidence for the expression of this transport system not only in the colon but also in the distal small intestine (Ganapathy, V. et al., Intestinal transport of peptides and amino acids. In Current Topics in Membranes. Ed. Barrett, K. E. and Donowitz, M., Vol.50, pp.379-412. Academic Press. (2001), Munck, L. K., Biochim.Biophys.Acta, 1241, 195-213, (1995)). The transport function has been shown to be present in the brush border membrane of the mucosal cells in the ileum (Ganapathy, V. et al., Intestinal transport of peptides and amino acids. In Current Topics in Membranes. Ed. Barrett, K. E. and Donowitz, M., Vol.50, pp.379-412. Academic Press. (2001), Munck, L. K., Biochim.Biophys.Acta, 1241, 195-213, (1995)). ATB^{0,+} mRNA is detectable in the present study only in the distal regions of the intestinal tract (ileum, cecum, and colon). The expression pattern of ATB^{0,+} mRNA along the longitudinal axis of the intestinal tract is interesting and of relevance to the potential use of this transporter as a delivery system for NOS inhibitors. To our knowledge, the restricted expression of ATB^{0,+} in the distal intestinal tract is unique among the amino acid transporters. Amino acids derived from the dietary proteins are absorbed mostly in the proximal small intestine and consequently the concentrations of amino acids in the distal regions of the intestinal tract are low. As a result, there will be little competition between NOS inhibitors and endogenous amino acids for transport via ATB^{0,+}. This will enhance the efficiency of intestinal absorption of NOS inhibitors.

There are two other amino acid transport systems in the intestinal brush border membrane which may participate in the uptake of NOS inhibitors from the intestinal lumen. These are system y⁺ and system b^{0,+}. The ability of system y⁺ to transport cationic NOS inhibitors has been well established. In contrast, there is very little information available on the ability of system b^{0,+} to transport NOS inhibitors. Since this transport system is able to interact with zwitterionic as well as cationic amino acids, the present inventors predict that this system can handle zwitterionic as well as cationic NOS inhibitors. Our recent studies have indeed demonstrated that system b^{0,+} is at least partly responsible for the uptake of the zwitterionic NOS inhibitor L-NNA across the intestinal brush border membrane (Hatanaka, T. et al., Pharmaceut.Res., 16, 1770-1774, (1999)). However, both system y⁺ and system b^{0,+} are not driven by any ion gradient. Therefore, the present inventors speculate that ATB^{0,+}, with its energetic coupling to transmembrane gradients of Na⁺ and Cl⁻, is likely to be much more efficient than system y⁺ and system b^{0,+} in the uptake of NOS inhibitors from the lumen into the intestinal and colonic absorptive cells.

The oral bioavailability of NOS inhibitors will depend not only on the existence of entry routes for these compounds in the intestinal and colonic brush border membrane but also on the existence of exit routes in the basolateral membrane. There are two amino acid transport systems in the basolateral membrane of the intestinal tract that may be of relevance to the exit of NOS inhibitors from the intestinal and colonic absorptive cells into the blood. These are system L and system y⁺L (Ganapathy, V. et al., Intestinal transport of peptides and amino acids. In Current Topics in Membranes. Ed. Barrett, K. E. and Donowitz, M., Vol.50, pp.379-412. Academic Press. (2001)). The NOS inhibitors that are absorbed into the intestinal and colonic epithelial cells via ATB^{0,+}, system y⁺, and system b^{0,+}, can exit these cells across the basolateral membrane via systems L and y⁺L.

The present studies may also be of clinical relevance to the management of intestinal and colonic inflammation with NOS inhibitors. There is convincing evidence for the induction of NOS II in the intestinal and colonic epithelial cells during inflammation (Tepperman, B. L. et al., Am.J.Physiol., 265, G214-G218, (1993), Singer, I. I. et al., 1996. Gastroenterology, 111, 871-875, (1996)). Nitric oxide plays an important role in the normal physiological function of the intestinal tract and also in pathological conditions such as bacterial sepsis and inflammatory bowel disease (Stensen, W. F., Gastrointestinal inflammation. In, Textbook of Gastroenterology (ed. Yamada, T.). Lippincott Williams & Wilkins, Philadelphia. pp. 123-140, (1999)). It is of interest to note that the inflammatory bowel diseases ulcerative colitis and Crohn's disease involve primarily colon and/or ileum, the sites at which ATB^{0,+} is principally expressed in the intestinal tract. The idea of using ATB^{0,+} as the delivery system for NOS inhibitors is particularly appealing for several reasons with respect to the clinical management of inflammatory bowel disease in which there is an induction of NOS II in the intestinal and colonic epithelial cells. ATB^{0,+} is a highly concentrative transporter and therefore the NOS inhibitors will be absorbed very effectively into the intestinal and colonic epithelial cells and accumulated inside the cells at high concentrations. This will result in an effective means of inhibiting NOS II in these cells. Furthermore, NOS inhibitors in the intestinal lumen will compete with arginine, the substrate for NOS II, for transport into the cells via ATB^{0,+} and thus reduce the availability of arginine for NOS II activity. Thus, ATB^{0,+} will allow NOS inhibitors to get into the cells in place of arginine. This will result in a very effective inhibition of NOS II activity, both by reducing the availability of arginine, the NOS II substrate, and by increasing the intracellular concentration of NOS inhibitors.

2.Na⁺- and Cl⁻-Coupled Active Transport of Carnitine by the Amino Acid Transporter ATB^{0,+}

Methods

Carnitine Transport via Mouse ATB^{0,+} in a Mammalian Cell Expression System.

The present inventors cloned a full-length functional ATB^{0,+} from a mouse colon cDNA library (GenBank accession no. AF320226). The cloned transporter was functionally expressed in human retinal pigment epithelial (HRPE) cells using the vaccinia virus expression technique (Wu, X et al., Biochemical and Biophysical Research Communications, 246,589-595, (1998), Wu, X., et al., Journal of Pharmacology and Experimental Therapeutics, 290, 1482-1492, (1999)). Initial studies of the interaction of carnitine with mouse ATB^{0,+} in HRPE cells were done by assessing the ability of carnitine to compete with glycine for transport via ATB^{0,+}. Subsequent studies were carried out using [³H]-carnitine to assess directly the transport of carnitine via ATB^{0,+}. Transport measurements were made in vector-transfected cells and in mouse ATB^{0,+} cDNA-transfected cells in parallel using 24-well culture plates. Incubations of the cells with radiolabeled substrates were carried out at 37° C. for 15 min. The composition of the transport buffer was 25 mM Hepes/Tris (pH 7.5), containing 140 mM NaCl, 5.4 mM KCl, 1.8 mM CaCl₂, 0.8 mM MgSO₄, and 5 mM glucose. cDNA-specific transport was calculated by adjusting for the transport in vector-transfected cells. [³H]-Glycine, [³H]-carnitine, acetyl-[³H]-carnitine and propionyl-[³H]-carnitine were purchased from Moravek Biochemicals (Brea, Calif., USA).

Carnitine Transport via Mouse ATB^{0,+} in the X. laevis oocyte Expression System.

Mature oocytes from X. laevis were isolated by treatment with collagenase A (1.6 mg/ml). Oocytes were manually defolliculated and then used for injection with mouse ATB^{0,+} cRNA or water (Fei, Y. J. et al., Journal of Biological Chemistry, 275, 23707-23717, (2000)). cRNA was synthesized using the mMESSAGE mMACHINE™ kit (Ambion, Austin, Tex., USA). The transport of carnitine via mouse ATB^{0,+} in oocytes was monitored electrophysiologically using the two-microelectrode voltage-clamp technique (Fei, Y. J. et al., Journal of Biological Chemistry, 275, 23707-23717, (2000)). The membrane potential was held steady at −50 mV. Oocytes were perifused with carnitine and the induced current was monitored. The induced current was taken as the measure of transport rate. The composition of the perifusion buffer was 10 mM Hepes/Tris (pH 7.5), containing 100 mM NaCl, 2 mM KCl, 1 mM MgCl₂, and 1 mM CaCl₂.

Data Analysis.

Experiments were repeated at least three times. Results are given as means±S. E. The kinetic parameters, Michaelis-Menten constant (Kₜ) and maximal velocity (Vₘₐₓ), were calculated by fitting the ATB^{0,+}-specific transport data to the Michaelis-Menten equation describing a single saturable transport system. Na⁺- and Cl⁻-activation kinetics were analyzed by fitting the ATB^{0,+}-specific transport data to the Hill equation for the determination of K_{0.5} values for Na⁺ and Cl⁻ (concentration of Na⁺ or Cl⁻ necessary for half-maximal activation) and the Hill coefficient (n^{H}, number of Na⁺ or Cl⁻ ions involved in the activation process).

Results

Analysis of Carnitine Transport via Mouse ATB^{0,+} in the Mammalian Cell Expression System.

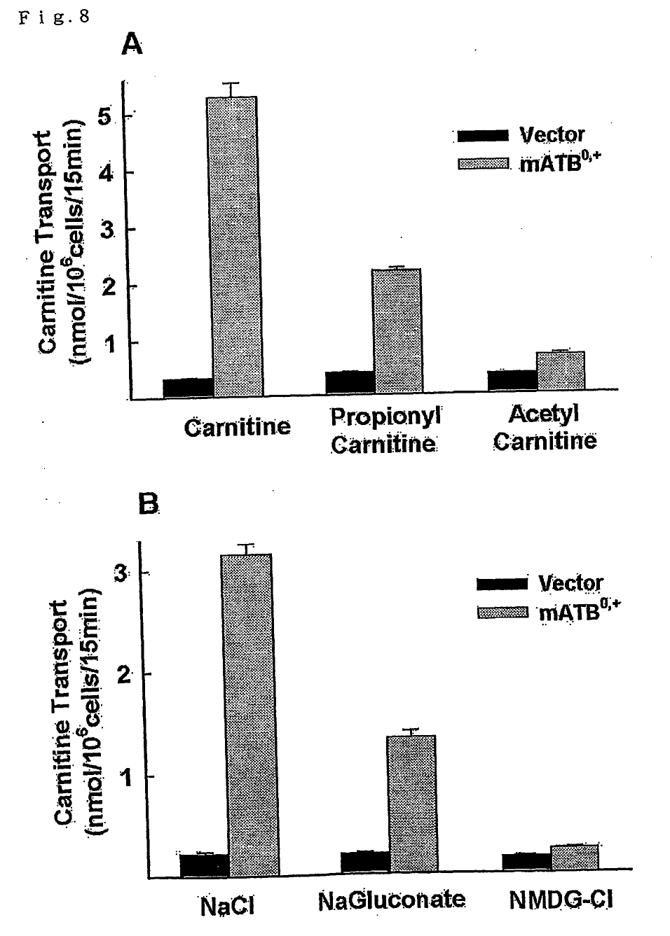

There is a significant structural similarity between carnitine and γ-aminobutyrate as well as between carnitine and betaine. Carnitine is a derivative of γ-aminobutyrate with an addition of a hydroxyl group at the β carbon and the substitution of the amino group with the trimethylamino group. Because of the presence of the trimethylamino group at the terminal carbon atom, carnitine is also structurally similar to betaine. Since the primary structure of ATB^{0,+} is closely related to that of γ-aminobutyrate transporters and betaine transporter, the present inventors investigated whether ATB^{0,+} is capable of interacting with γ-aminobutyrate and betaine. In the same experiment, the present inventors also tested the ability of ATB^{0,+} to interact with carnitine and its acetyl and propionyl esters. In these studies, the transport function of ATB^{0,+} was monitored by measuring the transport of glycine in HRPE cells expressing the cloned mouse ATB^{0,+}. The expression of ATB^{0,+} in these cells increased the transport of glycine by 30-fold (FIG. 8A). The interaction of the transporter with the test compounds was investigated by assessing their ability to inhibit ATB^{0,+}-mediated glycine transport (FIG. 8B). These studies produced interesting, but quite unexpected, results. γ-Aminobutyrate and betaine showed little or no effect on ATB^{0,+}-mediated glycine transport. In contrast, carnitine and propionylcarnitine inhibited ATB^{0,+}-mediated glycine transport markedly. The IC₅₀ values (concentration of the compound at which the inhibition was 50%) for carnitine and propionylcarnitine were 0.6±0.1 and 0.9±0.1 mM, respectively. Acetylcarnitine was also able to inhibit ATB^{0,+}-mediated glycine transport, but surprisingly the inhibitory potency of this ester was much less than that of carnitine and its propionyl ester (IC₅₀ for acetylcarnitine was 15±3 mM).

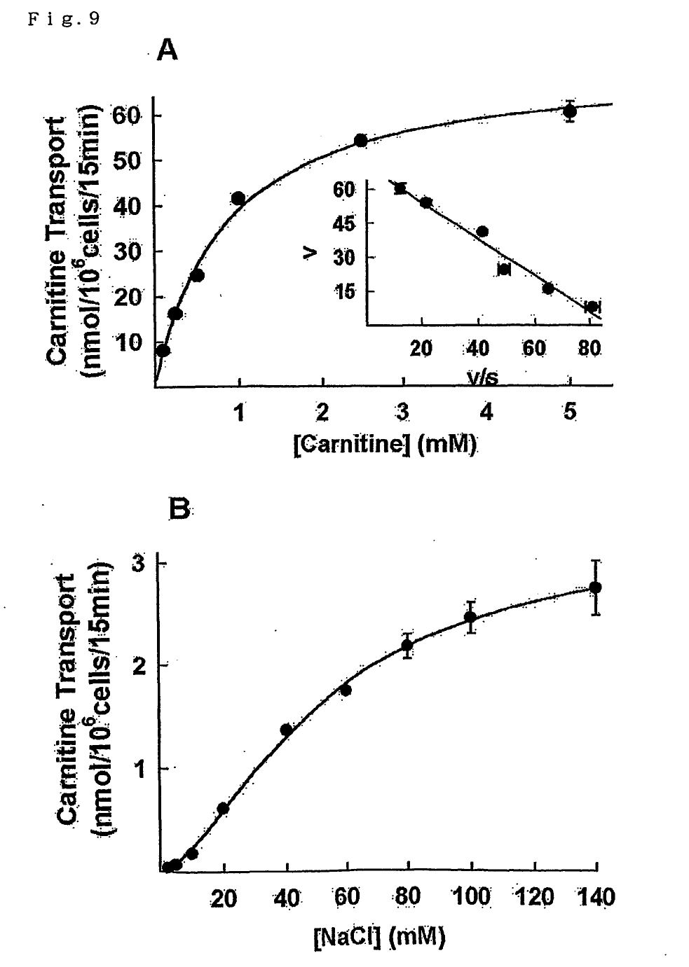

To determine whether or not carnitine and its esters are transportable substrates for ATB^{0,+}, the present inventors compared the transport of [³H]-carnitine and its esters between vector-transfected cells and ATB^{0,+} cDNA-transfected cells (FIG. 9A). Expression of ATB^{0,+} in HRPE cells induced the transport of carnitine (16-fold) and propionylcarnitine (6-fold) compared to transport in vector-transfected cells. The transport of acetylcarnitine was also increased by ATB^{0,+} expression but to a much smaller extent (2-fold). These data show that ATB^{0,+} recognizes carnitine, propionylcarnitine, and acetylcarnitine as transportable substrates. Since ATB^{0,+} is a member of the Na⁺- and Cl⁻-coupled transporter gene family, we investigated the influence of these two ions on the transport of carnitine mediated by ATB^{0,+} (FIG. 9B). The transport was completely abolished when Na⁺ in the uptake buffer was substituted with N-methyl-D-glucamine. Removal of Cl⁻ from the uptake buffer by substituting with gluconate reduced the transport by ˜60%. These results show that ATB^{0,+}-mediated carnitine transport is coupled to both Na⁺ and Cl⁻. The removal of Cl⁻ did not abolish the transport completely because of the possible release of Cl⁻ from the cells during transport measurements. The transporter interacts with Cl⁻ with high affinity and therefore the transporter is significantly active even at low concentrations of Cl⁻.

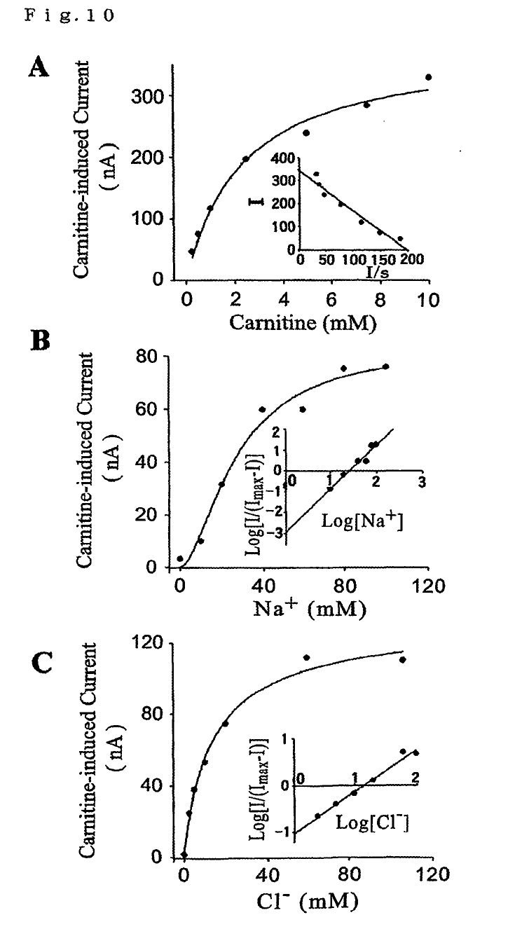

The transport of carnitine mediated by ATB^{0,+} was saturable (FIG. 10A). The values for the kinetic parameters Kₜ and Vₘₐₓ were 0.83±0.08 mM and 72±2 nmol/10⁶ cells/15 min. The relationship between ATB^{0,+}-mediated carnitine transport and Na⁺ concentration was sigmoidal (FIG. 10B). The K_{0.5} for Na⁺ was 54±4 mM and the Hill coefficient (n^{H}) was 1.6±0.1. The Cl⁻-activation kinetics were not investigated in this expression system because of the efflux of significant amounts of Cl⁻ from the cells during the experiment.

Analysis of Carnitine Transport via Mouse ATB^{0,+} in the X. laevis oocyte Expression System.

Transport of amino acid substrates via ATB^{0,+} is electrogenic (Sloan, J. L. and Mager, S., Journal of Biological Chemistry, 274, 23740-23745, (1999). To investigate the electrogenic nature of ATB^{0,+}-mediated carnitine transport, the present inventors employed the X. laevis oocyte expression system. When oocytes expressing the mouse ATB^{0,+} were perifused with carnitine, marked inward currents were detectable by the two-microelectrode voltage-clamp technique (˜300 nA at 10 mM carnitine) (data not shown). Under similar conditions, propionylcarnitine induced ˜200 nA currents. In contrast, acetylcarnitine was unable to induce any detectable currents. With carnitine and propionylcarnitine, the induced currents were absolutely dependent on the presence of Na⁺ as well as Cl⁻. Removal of either of the two ions abolished the currents completely.

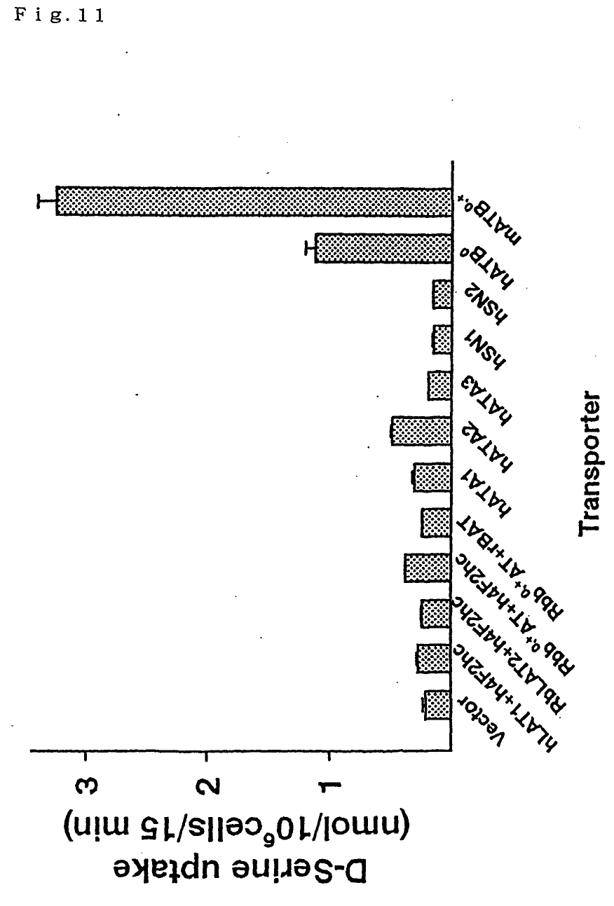

We analyzed the kinetic parameters of ATB^{0,+}-mediated carnitine transport using the carnitine-induced inward currents as the measure of the transporter function. The currents were saturable with increasing concentrations of carnitine (FIG. 11A). The K_{0.5} for carnitine was 1.8±0.4 mM. The Na⁺-activation kinetics of carnitine-induced currents showed a sigmoidal relationship (FIG. 11B). The K_{0.5} for Na⁺ was 25±4 mM and the Hill coefficient (n^{H}) was 1.9±0.5. Since the removal of Cl⁻ abolished completely the carnitine-induced currents, there was apparently no efflux of Cl⁻ from the oocytes under the experimental conditions. Therefore, we used this expression system to analyze the Cl⁻-activation kinetics. The relationship between carnitine-induced currents and Cl⁻ concentration was hyperbolic (FIG. 11C). The K_{0.5} for Cl⁻ was 15±5 mM and the Hill coefficient (n^{H}) was 0.9±0.2. These data show that the Na⁺:Cl⁻: carnitine stoichiometry for the ATB^{0,+}-mediated transport process was 2:1:1.

Discussion

To date, OCTN2 is the only transporter that has been shown to transport carnitine in an ion gradient-coupled manner (Wu, X. et al., Biochemical and Biophysical Research Communications, 246, 589-595, (1998), Wu, X., et al., Journal of Pharmacology and Experimental Therapeutics, 290, 1482-1492, (1999), Tamai, I. et al., Journal of Biological Chemistry, 273, 20378-20382, (1998)). The transport function of OCTN2 is dependent on the presence of Na⁺. Cl⁻ does not have any role in the function of this transporter. The transporter is however likely to be electrogenic due to the zwitterionic nature of carnitine and the coupling of the transport process with Na⁺ cotransport. OCTN2-mediated transport of carnitine is therefore energized by transmembrane Na⁺ gradient and membrane potential. The present studies describe the identification of a second ion gradient-coupled transporter for carnitine. ATB^{0,+} transports carnitine in a Na⁺- and Cl⁻-coupled manner. The transport process is electrogenic. Thus, the transport of carnitine via ATB^{0,+} is energized by transmembrane gradients of Na⁺ and Cl⁻ as well as membrane potential. The concentrative capacity of ATB^{0,+} for carnitine is much greater than that of OCTN2. However, ATB^{0,+} is a low-affinity transporter for carnitine (Kₜ=1-2 mM). In contrast, OCTN2 is a high-affinity transporter for carnitine (Kₜ=5-15 μM) (Tamai, I. et al., Journal of Biological Chemistry, 273, 20378-20382, (1998), Wu, X., et al., Journal of Pharmacology and Experimental Therapeutics, 290, 1482-1492, (1999)). The concentrations of carnitine in blood are in the range of 30-50 μM and therefore OCTN2 is more important that ATB^{0,+} for cellular uptake of carnitine in most tissues under physiological conditions. Interestingly, the tissue distribution of the two transporters is quite different. OCTN2 is expressed in most tissues whereas ATB^{0,+} is expressed primarily in the mammary gland, lung, and intestinal tract. ATB^{0,+} is likely to play a significant role in tissues in which it is expressed. Recent studies with JVS mice, which have a genetic defect in OCTN2 transport function, have shown that the intestinal absorption of carnitine is reduced only about 50% due to the defect (Yokogawa, K. et al., Journal of Pharmacology and Experimental Therapeutics 289, 224-230, (1999)). The findings that defects in OCTN2 function do not eliminate intestinal absorption completely suggest that some additional, hither to unidentified, transporters participate in the intestinal absorption of carnitine. Since ATB^{0,+} is expressed in the intestinal tract, it is possible that this transporter, along with OCTN2, participates in the intestinal absorption of carnitine. Furthermore, ATB^{0,+} is expressed not only in the small intestine but also in the colon. The present studies were done with ATB^{0,+} cloned from mouse colon. Micorbial flora utilize carnitine as a carbon source (Rebouche, C. J. and Seim, H., Annual Review of Nutrition, 18, 39-61, (1998)). Therefore, the present inventors speculate that ATB^{0,+} in the colon may play a role in the absorption of carnitine and thus compete with colonic bacteria for carnitine in the lumen. Similarly, ATB^{0,+} in the mammary gland may participate in the secretion of carnitine into milk. In addition, the transport of carnitine via ATB^{0,+} is likely to be very relevant to carnitine homeostasis in patients with genetic defects in OCTN2.

OCTN2 and ATB^{0,+} differ not only in their affinity and driving forces but also in their substrate specificity. OCTN2 transports carnitine, acetylcarnitine and propionylcarnitine with comparable affinity (Wu, X., et al., Journal of Pharmacology and Experimental Therapeutics, 290, 1482-1492, (1999)). In contrast, ATB^{0,+} transports only carnitine and propionylcarnitine. The transporter shows very low affinity for acetylcarnitine. Acetylcarnitine is the predominant acylcarnitine ester inside the cell as well as in the circulation. It is a key intermediate in anabolic and catabloic pathways of metabolism. The differential affinity of OCTN2 and ATB^{0,+} for acetylcarnitine may have physiological implications.

3.Na⁺- and Cl⁻-Coupled Active Transport of D-Amino Acids by the Amino Acid Transporter ATB^{0,+}

Methods

Methods are shown in table3 and BRIEF DESCRIPTION OF THE DRAWINGS (FIG. 11) briefly

Results & Discussion

The present inventors evaluated the function of 10 different amino acid transporter clones with respect to transport of D-serine in heterologous expression systems (FIG. 11). All of these transporters are expressed in the intestinal tract. The list of the transporters tested include three energy-independent, heterodimeric facilitative transporters (L1, L2, and b^{0,+}), three subtypes of the Na⁺-coupled system A (ATA1, ATA2, and ATA3), two subtypes of the Na⁺- and H⁺-coupled system N (SN1 and SN2), the Na⁺-coupled system ATB⁰, and the Na⁺- and Cl⁻-coupled system ATB^{0,+}. Among the facilitative transporters, system L1 (LAT1/4F2hc) and b^{0,+} (b^{0,+}AT/4F2hc) showed significant ability to transport D-serine. The LAT2/4F2hc and b^{0,+}AT/rBAT complexes did not exhibit detectable D-serine transport activity. Among the subtypes of system A, ATA1 and ATA2 showed D-serine transport activity, but ATA3 did not. SN1 and SN2 did not transport D-serine. ATB⁰ was able to transport D-serine to a marked extent, its transport activity being much higher than that of systems L1, b^{0,+}, ATA1, and ATA2. The ability to transport D-serine was the highest for the Na⁺- and Cl⁻-coupled transporter ATB^{0,+}. ATB^{0,+} belongs to the gene family of neurotransmitter transporters whose transport function is energized by multiple driving forces, namely a Na⁺ gradient, a Cl⁻ gradient, and membrane potential. The functional activities of ATB⁰ and ATB^{0,+} have been demonstrated in the brush border membrane of the intestinal epithelial cells, and therefore it is likely that these two transporters mediate the active absorption of D-serine from the lumen into the mucosal cells. Based on the energetics of these two transport systems, ATB^{0,+} is expected to play the leading role in this process.

To determine whether ATB^{0,+} is capable of transporting other D-amino acids, two different approaches were used (Table 3).

TABLE 3
Transport of L-amino acids versus D-amino acids via ATB^{0,+}

| | mATB^{0,+}-Specific | | Current in | |
|---|---|---|---|---|
| | [³H]-Glycine Transport | | X. laevis oocytes | |
| | in HRPE cells (% control) | | (nA) | |
| Amino Acid | L-isomer | D-isomer | L-isomer | D-isomer |
| Control | 100 ± 6 | 100 ± 6 | | |
| Alanine | 6 ± 1 | 10 ± 1 | 650 ± 86 | 402 ± 39 |
| Serine | 10 ± 1 | 14 ± 1 | 470 ± 42 | 267 ± 23 |
| Methionine | 3 ± 0 | 14 ± 1 | 278 ± 26 | 213 ± 15 |
| Leucine | 3 ± 1 | 13 ± 1 | 187 ± 14 | 161 ± 25 |
| Tryptophan | 4 ± 1 | 7 ± 1 | 112 ± 4 | 157 ± 23 |
| Threonine | 19 ± 2 | 47 ± 3 | 233 ± 12 | 41 ± 5 |
| Histidine | 9 ± 1 | 35 ± 2 | 410 ± 64 | 29 ± 6 |
| Phenylalanine | 3 ± 0 | 26 ± 2 | 275 ± 58 | 24 ± 24 |
| Glutamine | 13 ± 1 | 77 ± 4 | 453 ± 53 | 16 ± 11 |
| Asparagine | 15 ± 1 | 95 ± 7 | 394 ± 67 | 3 ± 2 |
| Lysine | 18 ± 1 | 86 ± 5 | 321 ± 34 | 9 ± 9 |
| Arginine | 25 ± 1 | 93 ± 6 | 339 ± 35 | 6 ± 1 |
| Valine | 7 ± 1 | 87 ± 4 | 310 ± 24 | 4 ± 3 |
| Isoleucine | 4 ± 1 | 85 ± 5 | 117 ± 16 | 4 ± 4 |

Transport of [³H]-glycine (10 μM) was measured in vector-transfected HRPE cells and in mATB^{0,+} cDNA-transfected HRPE cells. Unlabeled amino acids were used at a concentration of 2.5 mM. cDNA-specific transport was calculated by subtracting the transport in vector-transfected cells from the transport in mATB^{0,+} cDNA-transfected cells. Values are percent of control transport. Data (means±SEM from four separate determinations) represent only cDNA-specific transport. mATB^{0,+} was also expressed in X. laevis oocytes by injecting mATB^{0,+} cRNA, and the inward currents induced by amino acids (1 mM) were measured using the two-microelectrode voltage clamp technique. The perifusion medium contained NaCl (pH 7.5). Data represent means±SEM from three different batches of oocytes.

In the first approach, the transport function of ATB^{0,+} was measured in a mammalian cell heterologous expression system using glycine as the substrate and the ability of L- and D-enantiomers of various amino acids to inhibit this transport function was compared. AU neutral and cationic amino acids tested were potent inhibitors of ATB^{0,+}-mediated glycine transport when present as L-enantiomers. In the case of D-enentiomers, only alanine, serine, methionine, leucine and tryptophan were potent inhibitors. The extent of inhibition was comparable between L-enantiomers and D-enantiomers for these five amino acids. In contrast, the D-enantiomers of threonine, histidine, phenylalanine, and glutamine were much less effective than the corresponding L-enantiomers as inhibitors. Asparagine, lysine, arginine, valine, and isoleucine were almost totally ineffective as inhibitors when present as D-enantiomers even though the corresponding L-enantiomers were potent inhibitors. Since the inhibition does not necessarily mean that the inhibitors are translocated across the membrane via the transporter, the transport of L- and D-enantiomeric forms of these amino acids was assessed directly in Xenopus laevis oocytes expressing ATB^{0,+} heterologously. This was done using the two-microelectrode voltage-clamp technique and monitoring the amino acid-induced inward currents. The L-enantiomers of all amino acids tested induced inward currents, indicating their transport via ATB^{0,+}. In the case of D-enantiomers, only alanine, serine, methionine, leucine, and tryptophan induced currents. Threonine, histidine, phenylalanine, and glutamine produced small but significant currents whereas the remaining amino acids did not produce currents. These results with the X. laevis oocyte expression system corroborate the results with the mammalian cell expression system. These data show that ATB^{0,+} is capable of transporting all neutral and cationic amino acids when presented as the L-enantiomers. But, the transporter recognizes only alanine, serine, methionine, leucine, and tryptophan in their D-enantiomeric form as transportable substrates.

4.Na⁺- and Cl⁻-Coupled Active Transport of Phenylglycine and its Derivative by the Amino Acid Transporter ATB^{0,+}

Methods

Materials.

[³H]-Glycine was purchased from Moravek (Brea, Calif.) and [¹⁴C]-L-phenylglycine was purchased from American Radiolabeled Chemicals, Inc. (St. Louis, Mo.). [³H]-L-Glutamine and [³H]-L-alanine were obtained from Dupont-New England Nuclear (Boston, Mass.) and Amersham Pharmacia Biotech(Piscataway, N.J.), respectively.

Functional Expression of ATB^{0,+}, ATB⁰ and b^{0,+} AT in HRPE cells.

This was done using the vaccinia virus expression system. Transport measurements were made at 37° C. for 15 min with radiolabeled amino acids as substrates. The transport buffer was 25 mM Hepes/Tris (pH 7.5) containing 140 mM NaCl, 5.4 mM KCl, 1.8 mM CaCl₂, 0.8 mM MgSO₄, and 5 mM glucose. Endogenous transport activity was always determined in parallel using cells transfected with vector alone. cDNA-specific transport was calculated by adjusting for the endogenous activity.

Functional Expression of ATB^{0,+} in the X. laevis oocyte Expression System.

Mature oocytes from X. laevis were isolated by treatment with collagenase A (1.6 mg/ml). Oocytes were manually defolliculated and then used for injection with mouse ATB^{0,+} cRNA or water (Fei, Y. J. et al., Journal of Biological Chemistry, 275, 23707-23717, (2000)). cRNA was synthesized using the mMESSAGE mMACHINE™ kit (Ambion, Austin, Tex., USA). The transport of compounds via mouse ATB^{0,+} in oocytes was monitored electrophysiologically using the two-microelectrode voltage-clamp technique (Fei, Y. J. et al., Journal of Biological Chemistry, 275, 23707-23717, (2000)). The membrane potential was held steady at −50 mV. Oocytes were perifused with each compound and the induced current was monitored. The induced current was taken as the measure of transport rate. The composition of the perifusion buffer was 10 mM Hepes/Tris (pH 7.5), containing 100 mM NaCl, 2 mM KCl, 1 mM MgCl₂, and 1 mM CaCl₂.

### Synthesis of (S)-2-(3-Benzyloxycarbonylaminophenyl)-2-tert-butoxycarbonylaminoacetic acid tert-butyl ester

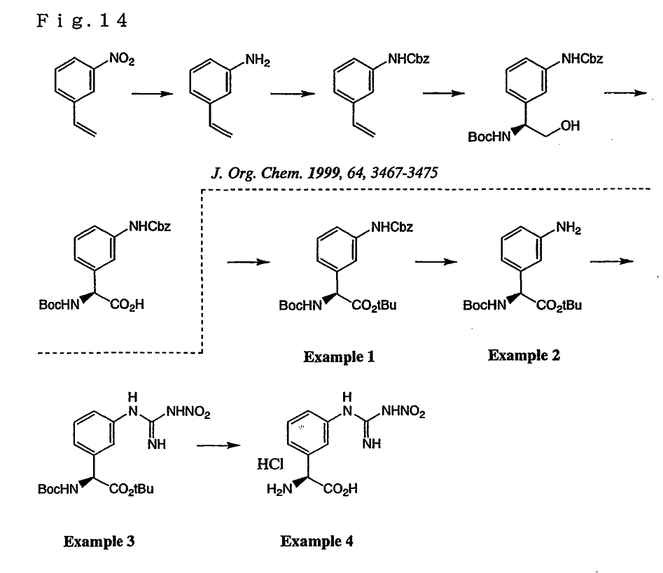

### FIG. 14 EXAMPLE 1

A mixture of (S)-2-(3-Benzyloxycarbonylaminophenyl)-2-tert-butoxycarbonyl-aminoacetic acid (1.0 g), tert-Butyl 2,2,2-trichloroacetimidate (1.0 g) and BF₃-Et₂O (a drop) in CH₂Cl₂ (30 mL) was stirred overnight at room temperature. The reaction mixture was concentrated in vacuo and the crude product was purified by column chromatography (3:1 hexane:EtOAc) to afford the titled compound (800 mg). ¹H NMR (CDCl₃, 400 MHz) δ 7.33 (m, 9H), 5.59 (brm, 1H), 5.19 (s, 2H), 5.18 (brm, 1H), 1.42 (m, 18H)

### Synthesis of (S)-2-(3-Aminophenyl)-2-tert-butoxycarbonylaminoacetic acid tert-butyl ester

### FIG. 14 EXAMPLE 2

A mixture of (S)-2-(3-Benzyloxycarbonylaminophenyl)-2-tert-butoxycarbonyl-aminoacetic acid tert-butyl ester (800 mg) and 10% Pd—C in EtOH (30 mL) was stirred under H₂. The mixture was filtered to remove the catalyst, and the filtrate was concentrated in vacuo to give the titled compound (650 mg). ¹H NMR (CDCl₃, 400 MHz) δ 7.12-7.08 (m, 1H), 6.73-6.59 (m, 3H), 5.48 (m, 1H), 5.08 (d, J=7.6 Hz, 1H), 3.67 (brs, 2H), 1.43 (s, 9H), 1.39 (s, 9H)

### Synthesis of (S)-2-tert-Butoxycarbonylamino-2-(3-(N′-nitroguanidino)phenyl)acetic acid tert-butyl ester

### FIG. 14 EXAMPLE 3

A mixture of (S)-2-(3-Aminophenyl)-2-tert-butoxycarbonylaminoacetic acid tert-butyl ester (2.6 g), N-Methyl-N′-nitro-N-nitrosoguanidine (2.2 g) and Et₃N—AcOH/MeCN (1M, 8.0 mL) in MeCN (50 mL) was stirred at 50° C. for 3 days in an airtight container. The reaction mixture was concentrated in vacuo and the crude product was purified by column chromatography (2:1 hexane:EtOAc and 15:1 CHCl₃:MeOH) to afford the titled compound (900 mg). ¹H NMR (CDCl₃, 400 MHz) δ 9.90 (brs, 1H), 7.48-7.27 (m, 4H), 5.80 (d, J=6.4 Hz, 1H), 5.19 (d, J=6.4 Hz, 1H), 1.40 (s, 18H)

### Synthesis of (S)-2-Amino-2-(3-(N′-nitroguanidino)phenyl)acetic acid hydrochloride

### FIG. 14 EXAMPLE 4

A mixture of (S)-2-tert-Butoxycarbonylamino-2-(3-(N′-nitroguanidino)phenyl)acetic acid tert-butyl ester (800 mg) and H₂O (1 mL) in HCl/dioxane (4M, 5 mL) was stirred overnight at room temperature. The mixture was concentrated in vacuo, dissolved in H₂O (5 mL), passed through a 5 mL solid-phase extracter (Waters ODS), and lyopholyzed to yield the titled compound (550 mg). The obtained compound was dissolved in H₂O (5 mL) and was applied to a chromatography column preapplied with 15 mL DOWEX 50W X8 ion-exchange resin (Na+ form). The column was washed with water and rinsed with 2% aqueous ammonia to ensure complete elution of (S)-2-Amino-2-(3-N′-nitroguanidino)phenyl)acetic acid. The ammonia eluent was lyopholyzed and the resulting residue was dissolved in H₂O (10 mL) again. (−)-10-Camphorsulfonic acid (460 mg) was added to the solution and was heated to 50° C. The homogeneous mixture was cooled to 4° C. and was stood overnight at the same temperature. The mixture was filterd to remove precipitated solid, and the filtrate was applied to a chromatography column preapplied with 15 mL DOWEX 50W X8 ion-exchange resin (Na+ form). The column was washed with water and rinsed with 2% aqueous ammonia to ensure complete elution of (S)-2-Amino-2-(3-(N′-nitroguanidino)phenyl)acetic acid. The ammonia eluent was lyopholyzed. 1M HCl (2 mL) and H₂O (10 mL) were added to the residue and the mixture was lyopholyzed to yield the titled compound (350 mg, 98% ee). ¹H NMR (DMSO-d₆, 400 MHz) δ 10.15 (s, 1H), 8.86 (m, 3H), 8.41 (brs, 2H), 7.43-7.17 (m, 4H), 5.07 (m, 1H)

Results and Discussion

The amino acid specificity of ATB^{0,+} was studied by assessing the ability of a variety of amino acids including L-phenylglycine to compete with [³H]-glycine (60 nM) for the transport process mediated by the cloned transporter (table 4).

TABLE 4
Amino Acid Substrate Specificity of Mouse ATB^{0,+}

| | mATB^{0,+}-Specific | |
|---|---|---|
| Unlabeled | [³H]-Glycine Transport | |
| Amino Acid | pmol/10⁶ cells/15 min | % |
| Control | 37.27 ± 1.20 | 100 |
| Glycine | 1.88 ± 0.06 | 5 |
| Alanine | 1.04 ± 0.05 | 3 |
| Cysteine | 1.11 ± 0.20 | 3 |
| Serine | 1.99 ± 0.09 | 5 |
| Threonine | 3.91 ± 0.30 | 11 |
| Proline | 9.97 ± 0.57 | 27 |
| Histidine | 1.31 ± 0.10 | 4 |
| Glutamine | 2.39 ± 0.18 | 6 |
| Asparagine | 3.25 ± 0.23 | 9 |
| Leucine | 0.27 ± 0.03 | 1 |
| Isoleucine | 0.27 ± 0.02 | 1 |
| Phenylalanine | 0.56 ± 0.03 | 2 |
| Tryptophan | 0.37 ± 0.06 | 1 |
| Arginine | 7.14 ± 0.35 | 19 |
| Lysine | 3.52 ± 0.28 | 9 |
| Aspartate | 32.01 ± 1.29 | 86 |
| Glutamate | 33.47 ± 0.91 | 90 |
| MeAIB | 36.21 ± 4.80 | 97 |
| Phenylglycine | 0.03 ± 0.02 | 0 |

Transport of [³H]-glycine (60 nM) was measured in vector-transfected HRPE cells and in mATB^{0,+} cDNA-transfected HRPE cells at 37° C. for 15 min in the presence of NaCl (pH 7.5). Unlabeled amino acids were used at a concentration of 2.5 mM. cDNA-specific transport was calculated by subtracting the transport in vector-transfected cells from the transport in mATB^{0,+} cDNA-transfected cells. Data (means±SEM from four separate determinations) represent only cDNA-specific transport.

At a concentration of 25 mM, all zwitterionic and cationic amino acids tested inhibited the transport of [³H]-glycine mediated by ATB^{0,+} (70 to 99%). L-Phenylglycine inhibited the transport of [³H]-glycine mediated by ATB^{0,+} completely (100%). This shows that L-phenylglycine has higher affinity against ATB^{0,+} than all the essential amino acids.

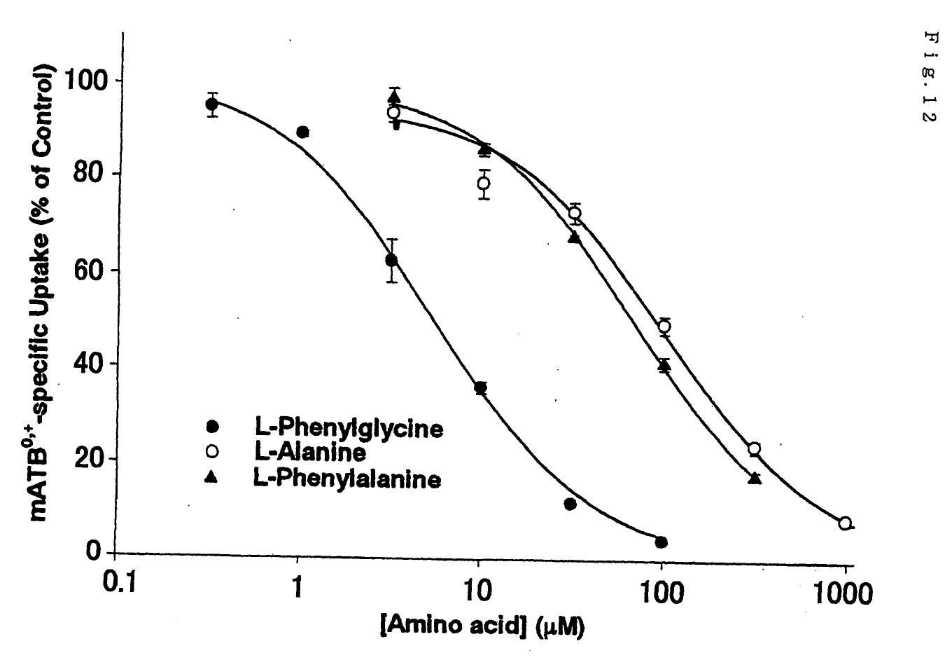

To quantify the difference of affinity for ATB^{0,+} between L-phenylglycine and essential amino acids, we compared the dose-response relationship for the inhibition of the transport of [³H]-glycine mediated by ATB^{0,+} with L-phenylglycine, L-alanine and L-phenylalanine (FIG. 12). The IC₅₀ values (i.e. concentration of the amino acid causing 50 % inhibition) calculated from these studies are given in Table 5. These IC₅₀ values are almost equal to Kᵢ values (i.e. inhibition constant) under the experimental conditions because the concentration of [³H]-glycine used in these competition is 57 nM, a value much lower than the known Kₜ values (>6 μM) of ATB^{0,+} for most amino acids. A comparison of these values indicates that the affinity of ATB^{0,+} for L-phenylglycine is 19 times and 13 times higher than the corresponding affinities for glycine and phenylalanine, respectively.

On intestinal brush border membranes (BBM), at least three transporters are supposed to transport L-phenylglycine; ATB^{0,+}, ATB⁰ and b^{0,+}AT. To prove that L-phenylglycine is the specific substrate for ATB^{0,+} in intestinal BBM, we compared the dose-response relationship for the inhibition of the transport of [³H]-alanine and [³H]-glutamine mediated by ATB⁰ and b^{0,+}AT, respectively, with L-phenylglycine, L-alanine and L-phenylalanine. Moreover, we calculated IC₅₀ values from these results (table 5).

TABLE 5
Comparison of Kᵢ values for the inhibition of transport mediated by
mATB^{0,+}, hATB⁰ and mb^{0,+} AT rBAT complex.

| | Kᵢ values | | |
|---|---|---|---|
| | | | mb^{0,+} AT rBAT |
| Amino Acid | mATB^{0,+} | hATB⁰ | complex |
| L-Phenylglycine | 5.3 ± 0.4 | 19.0 ± 5.5 | 283.3 ± 84.4 |
| L-Alanine | 101.4 ± 14.2 | 22.5 ± 4.2 | 803.7 ± 142.5 |
| L-Phenylalanine | 68.6 ± 2.4 | 4229 ± 999 | 586.5 ± 215.4 |

Uptake measurement were made as described in Table 1, except that the inhibition was assessed over a range of concentrations of unlabeled amino acids. Kᵢ values were calculated from the dose-response relationship for the inhibition of the uptake of 57 nM [³H]-glycine that was specific to mATB^{0,+}, 32 nM [³H]-Alanine that was specific to HATB⁰ and 36 nM [³H]-Arginine that was specific to the mb^{0,+} AT rBAT complex.

For L-phenylglycine, affinity of ATB^{0,+} is 4 times and 53 times higher than the corresponding affinities of ATB⁰ and b^{0,+}AT, respectively.

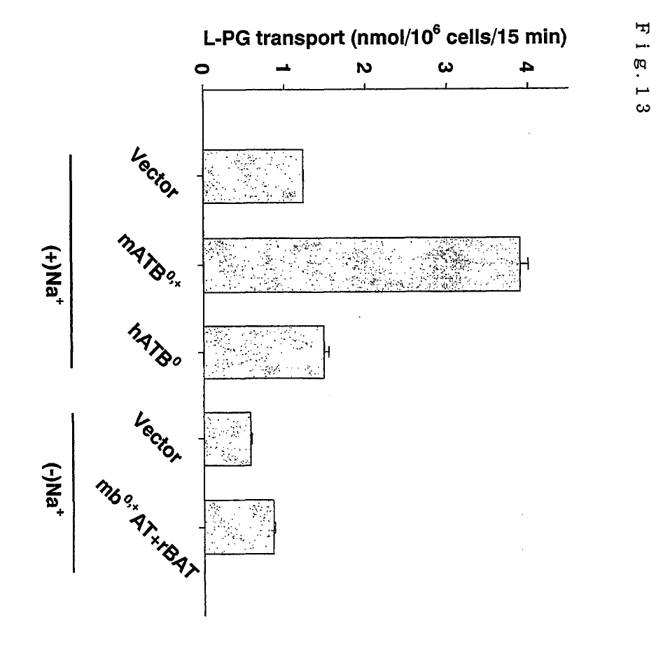

These results show that L-phenylglycine interacts with the substrate-binding site of the cloned mouse ATB^{0,+} with high affinity. However, these data suggest but do not prove that phenylglycine are transportable substrates for the transporter. It is possible that some compounds may block the transport by competing with glycine for binding to the substrate-binding site without itself being transported across the membrane. To determine whether or not these inhibitors are actually transportable substrates of the transporter, direct measurements of ATB^{0,+}-mediated transport of these inhibitors have to be carried out. Towards this goal, we used [¹⁴C]-L-phenylglycine as a substrate for ATB^{0,+} and studied its transport in HRPE cells expressing the cloned transporter (FIG. 13). The transport of L-phenylglycine in ATB^{0,+}-expressing cells was three times higher than in vector-transfected cells, demonstrating that L-phenylglycine is indeed a transportable substrate for this transporter. Moreover, cDNA-specific L-phenylglycine transport by ATB^{0,+} was ten times higher than the cDNA-specific transport by ATB⁰ and mb^{0,+}AT.

Taken together, these results show that L-phenylglycine is the most specific substrate for ATB^{0,+} among all we have tested. Phenylglycine-derivative drugs should be efficiently absorbed from intestine and delivered into the tissues expressing ATB^{0,+} abundantly, such as, colon, lung and mammal gland.

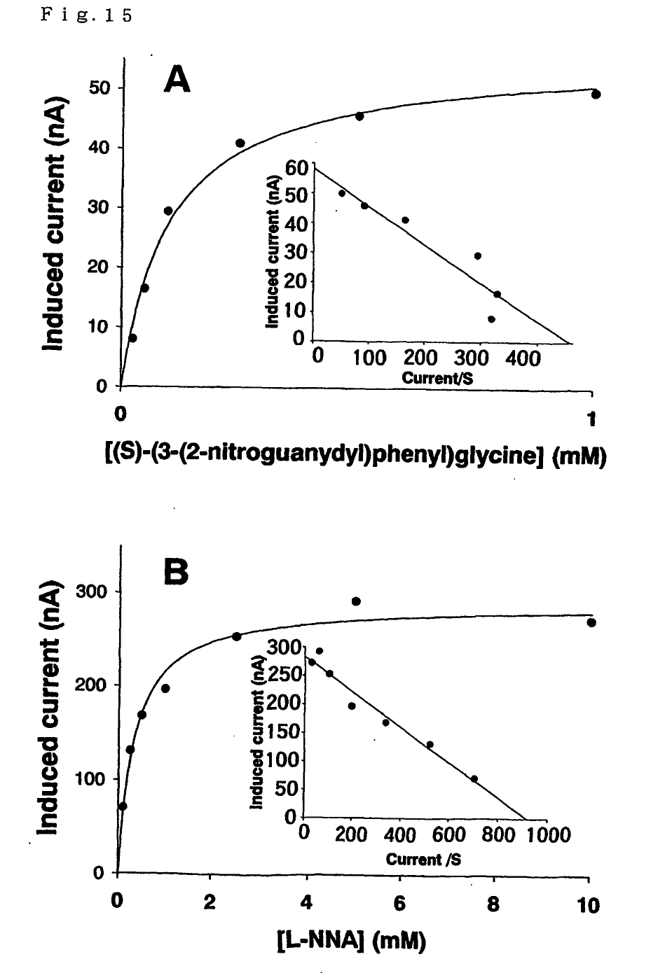

Moreover, the present inventors have synthesized the (S)-(3-(2-nitroguanydyl)phenyl)glycine as an example of L-phenylglycine derivatives (FIG. 14). This compound is supposed to be effective as a NOS inhibitor, because it is also a derivative of L-NNA, a potent NOS inhibitor. The present inventors used the X.laevis oocyte expression system to study the transport of this compound. Transport of this compound was monitored by inward currents induced by this compound at the various concentrations (FIG. 15A). The present inventors also monitored the currents of L-NNA to compare the affinities of both compounds (FIG. 15B). The Kₘ values for (S)-(3-(2-nitroguanydyl)phenyl)glycine and L-NNA are 106±1 and 348±5 μM, respectively. These data show the modification of chemical structure of L-NNA to the phenylglycine derivative increases the transport affinity to ATB^{0,+}. As the present inventors show in example 6, ATB^{0,+} is inducible in inflammatory condition, similar to iNOS. Taken together, the L-phenylglycine derivative NOS inhibitor, such as (S)-(3-(2-nitroguanydyl)phenyl)glycine should be effective for inflammation of the tissues expressing ATB^{0,+} abundantly, such as, colon, lung and mammal gland.

5. Evidence for the Potential use of ATB^{0,+} as a Delivery System for Amino Acid-Based Prodrugs

Methods

Transport Study

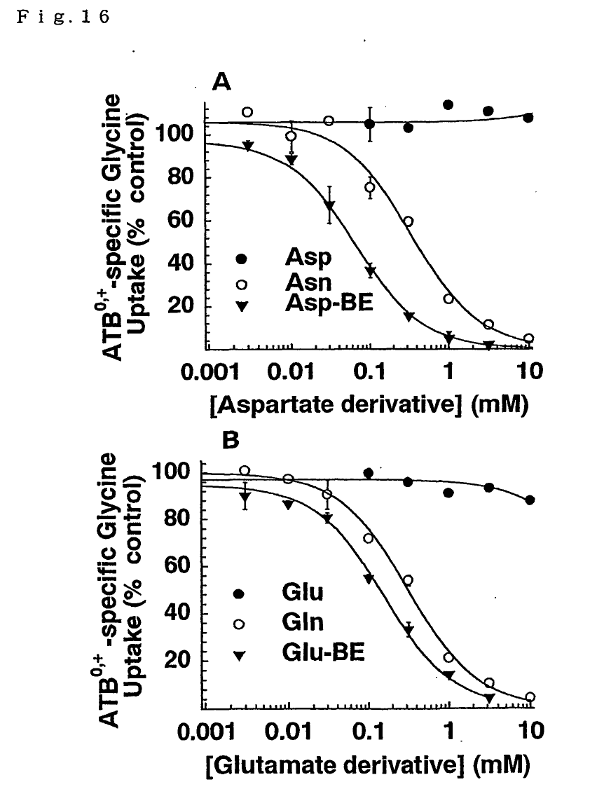

Methods are shown in table6 and BRIEF DESCRIPTION OF THE DRAWINGS (FIG. 16) briefly.

Synthesis of L-glutamate γ-ester of Acyclovir

Acyclovir and ammonium acetate were purchased from Sigma (St. Louis, Mo.). N-α-CBZ-L-glutamic acid γ-benzyl ester (Z-Glu-OBzl) was purchased from Nova biochem (San Diego, Calif.). 1-Ethyl-3-(3′-dimethylaminopropyl)carbodiimide (EDC), palladium 10 wt. % on activated carbon (Pd/C), 4-dimethylaminopyridine (DMAP), dimethylformamide (DMF), and dichloromethane (CH₂Cl₂) were purchased from Aldrich (Milwaukee, Wis.). Methanol (MeOH), and HPLC grade acetonitrile were from EM science (Gibbstown, N.J.). Ethanol (EtOH) was from AAPER alcohol and chemical company (Shelbyville, Ky.). Acyclovir (0.888 mmol) and Z-Glu-OBzl (2.22 mmol) were dissolved in DMF (6 ml), and EDC (426 mmol) and DMAP (2.22 mmol) were added to the solution. The solution was stirred for 18 h at room temperature. DMF was removed in vacuo, and the residue was chromatograghed on NH silica gel (Chromatorex DM-1020, Fuji Silysia Chemical LTD., Japan) using 1:10 to 1:5 MeOH—CH₂Cl₂ as the eluent to generate Acyclovir Z-Glu-OBzl γ ester.

Acyclovir Z-Glu-OBzl γ ester was dissolved in EtOH (1 ml), MeOH (1ml), and CH₂Cl₂ (1 ml), and added 10% Pd/C (20 mg). The mixture was stirred under hydrogen for 3 days. The mixture was filtered to remove the catalyst, and the solvent was removed in vacuo. The product was a white amorphous solid (120 mg). Overall yield: 38%. ¹H NMR (DMSOd₆) δ 2.00 (2H, m, CH₂), 3.14-4.10 (7H, m, 3×CH₂+CH), 5.34 (2H, m, CH₂), 6.78 (2H, bs, NH₂), 7.97 (1H, s aromatic-H), 7.39 (3H, bs,), 10.96 (1H, bs, NH); ESI-MS m/z calcd. 555.1, found 555.0 (MH)⁺.

The purity of the final product was confirmed by RP-HPLC using a Dionex analytical HPLC system ( Dionex corporation, Sunnyvale, Calif.) with an analytical column (Cyclobond I 2000, 4.6×100 mm, Advanced Separation Technologies Inc., Whippany, N.J.). The gradient for analytical RP-HPLC was as follows: (solvent A) 10% 5 mM aqueous ammonium acetate in acetonitrile, (solvent B) 90% 5 mM aqueous ammonium acetate in acetonitrile, 100:0 to 90:10 (solvent A: solvent B) over 3 min at 1 ml/min. Retention time was 1.42 min. with 96% purity. The molecular weight of the compound was determined by ESI-MS (Finnigan Mat LC-Q, Finnigan Corporation, San Jose, Calif.). The values are expressed as MH⁺.

Results and Discussion

Since aspartate and glutamate are not substrates for ATB^{0,+} but asparagine and glutamine are, the present inventors hypothesized that derivatives of aspartate and glutamate in which the carboxyl group in their side chains is substituted with different chemical moieties will become substrates for the transporter. To test this hypothesis, the present inventors assessed the ability of several such derivatives to compete with glycine for transport via ATB^{0,+} (Table 6). The present inventors found that the methyl and benzyl esters of aspartate and glutamate are potent inhibitors of glycine transport. At a concentration of 1 mM, these derivatives caused 80-100% inhibition, indicating that these derivatives are recognized by ATB^{0,+}. The hydroxamate, anilide, and naphthylamide derivatives are also moderately effective competing with glycine for transport via ATB^{0,+}. FIG. 16A describes the dose-response relationship for aspartate, asparagine, and the aspartate-β-benzyl ester and FIG. 16B describes the dose-response relationship for glutamate, glutamine, and glutamate-γ-benzyl ester. As expected, aspartate and glutamate are not effective inhibitors of ATB^{0,+}-mediated glycine transport. However, the amide derivatives (asparagine and glutamine) and the benzyl ester derivatives are potent inhibitors. Interestingly, the benzyl ester of aspartate (IC₅₀ value, 70±8 μM) is about 2-fold more potent that the benzyl ester of glutamate (IC₅₀ value, 156±12 μM), suggesting that ATB^{0,+} may interact with aspartate derivatives much better than with corresponding glutamate derivatives. Therefore, the present inventors speculate that if therapeutic drugs can be coupled to aspartate at the β-carboxyl group, the resultant derivatives may become excellent substrates for ATB^{0,+}.

TABLE 6

| | ATB^{0,+}-specific |
|---|---|
| Compound | glycine transport |
| (1 mM) | (% control) |
| 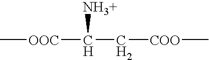 | 86 ± 3 |
| L-Aspartate | |
| 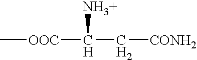 | 22 ± 0 |
| L-Asparagine | |
| 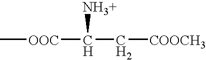 | 8 ± 1 |
| L-Aspartate β-methyl ester | |
| 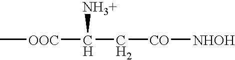 | 39 ± 1 |
| L-Aspartate β-hydroxamate | |
| 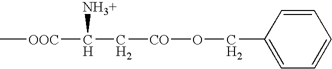 | 2 ± 1 |
| L-Aspartate β-benzyl ester | |
| 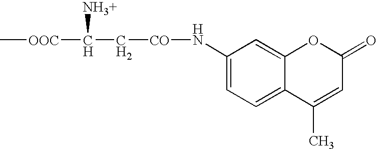 | *92 ± 5 |
| L-Aspartate | |
| β-(7-amido-4-methylcoumarin) | |
| 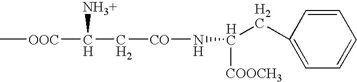 | 62 ± 3 |
| NβL-Aspartyl-L-phenylalanine | |
| methyl ester | |
| 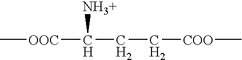 | 82 ± 2 |
| L-Glutamate | |
| 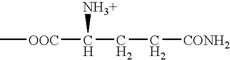 | 21 ± 1 |
| L-Glutamine | |
| 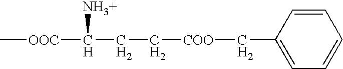 | 13 ± 1 |
| L-Glutamate γ-benzyl ester | |
| 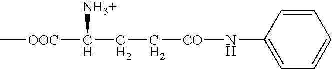 | 49 ± 1 |
| DL-Glutamate γ-anilide | |
| 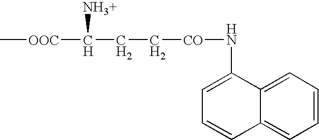 | 97 ± 2 |
| L-Glutamate γ-(α-napthylamide) | |
| 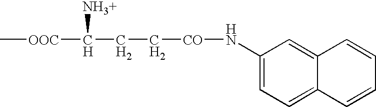 | *79 ± 4 |
| L-Glutamate γ-(β-napthylamide) | |

*0.25 mM

HRPE cells were transfected with either vector alone or mouse ATB^{0,+} cDNA and the functional expression was carried out by the vaccinia virus technique. Transport of [³H] glycine (10 μM) was measured in the presence and absence of various derivatives of aspartate and glutamate (1 mM). Data represent only cDNA-specific transport. Results are given as % of control transport measured in the absence of inhibitors.

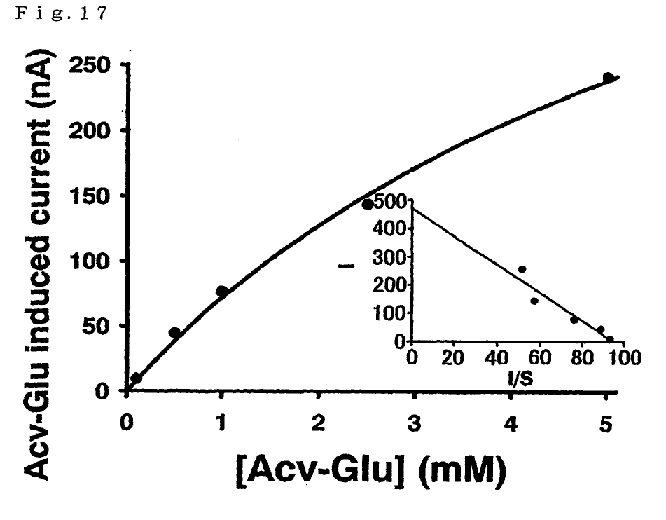

The parent drugs of these prodrugs are not real drugs. The present inventors have synthesized the L-glutamate γ-ester of acyclovir, a commercially available drug for virus infection, to study the practicality of these ideas. To assess this study, the present inventors used the X.laevis oocyte expression system. Transport of this prodrug was monitored by inward currents induced by this prodrug at the various concentration (FIG. 17). These data show the saturable transport of L-glutamate γ-ester of acyclovir by ATB^{0,+} directly.

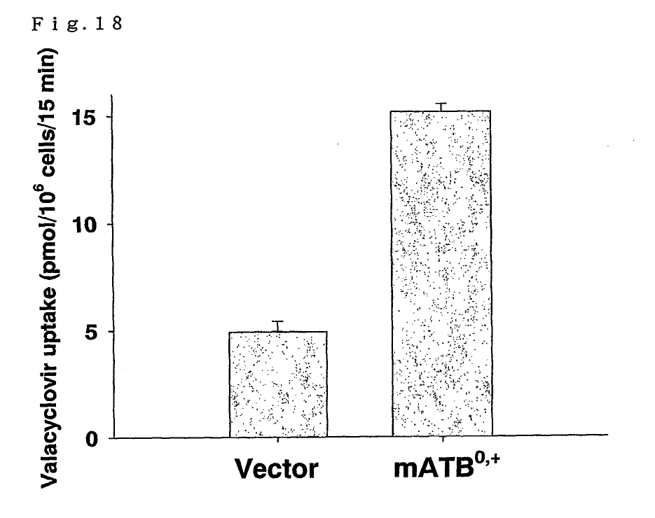

Moreover, the present inventors assessed the transport of L-valine α-ester of acyclovir (valacyclovir) to study the possibility of expansion of the substrate recognition of amino acid-based prodrugs. The present inventors studied [8-³H]-valacyclovir transport in HRPE cells transfected with mouse ATB^{0,+} cDNA (FIG. 18). As a result, the transport of [8-³H]-valacyclovir in ATB^{0,+}-expressing cells was three times higher than in vector-transfected cells, demonstrating that valacyclovir is indeed a transportable substrate for this transporter

6. Up-Regulation of ATB^{0,+} Expression in the Intestinal Tract Under Inflammatory Conditions

Methods

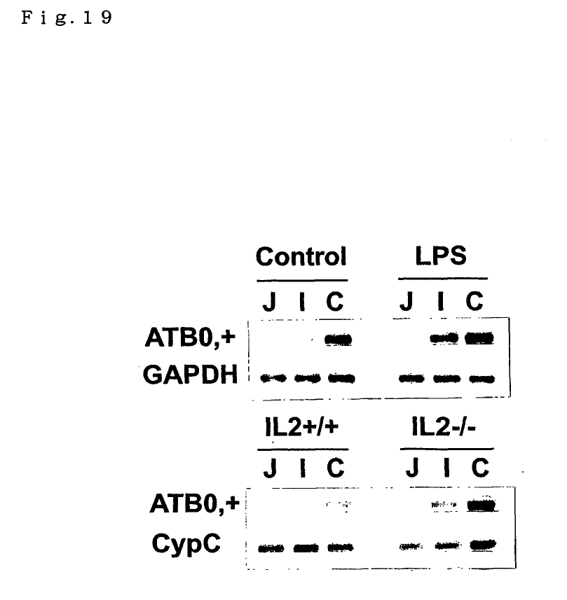

Methods are shown in BRIEF DESCRIPTION OF THE DRAWINGS (FIG. 19) briefly.

Results and Discussion

Since D-serine is an important modulator of glutamatergic neurotransmission and other D-amino acids may also have significant biological effects, it is of clinical relevance to know if there are any pathological conditions in which the intestinal absorption of D-amino acids may be altered. Therefore, the present inventors assessed the expression of ATB^{0,+} in the intestinal tract under inflammatory conditions that are known to affect intestinal function. The present inventors used two different animal models for this purpose. First, the present inventors used an animal model of sepsis in which sepsis was produced in mice by injection of bacterial lipopolysaccharide. This animal model has been used widely to study the influence of sepsis on intestinal morphology and function. Second, the present inventors used the interleukin-2 knockout mice as a model for colonic inflammation. IL-2^{-/-} mice develop severe ulcerative colitis within five to six weeks after birth. In the first model, mice injected with saline served as the control. In the second model, IL-2^{+/+} mice served as the control. The steady-state levels of ATB^{0,+} mRNA were examined by a semi-quantitative RT-PCR in the jejunum, ileum, and colon. Glyceraldehyde 3-phosphate dehydrogenase (GAPDH) mRNA was used as an internal control for the bacterial sepsis model. This mRNA was not unsuitable however as an internal control for the ulcerative colitis model because GAPDH mRNA was elevated several-fold in IL-2^{-/-} mice compared to IL-2^{+/+} mice. Therefore, the present inventors used cyclophilin C mRNA as an internal control for this animal model. After adjusting for the respective internal control, ATB^{0,+} mRNA was found to be increased 7-fold in the ileum and 2-fold in the colon in lipopolysaccharide -treated mice compared to saline-treated mice (FIG. 19). Similarly, the ATB^{0,+} mRNA levels increased 12-fold in the ileum and 5-fold in the colon in IL-2^{-/-} mice compared to IL-2^{+/+} mice (FIG. 19). These data show that inflammatory conditions lead to a marked increase in the expression of ATB^{0,+} in the ileum and colon. ATB^{0,+} mRNA was not detectable in the jejunum in any of the mice. The present inventors speculate that the intestinal absorption of bacteria-derived D-serine and other D-amino acids may be increased significantly under these inflammatory conditions.

7. Up-Regulation of ATB^{0,+} Expression in the Tumor Tissues

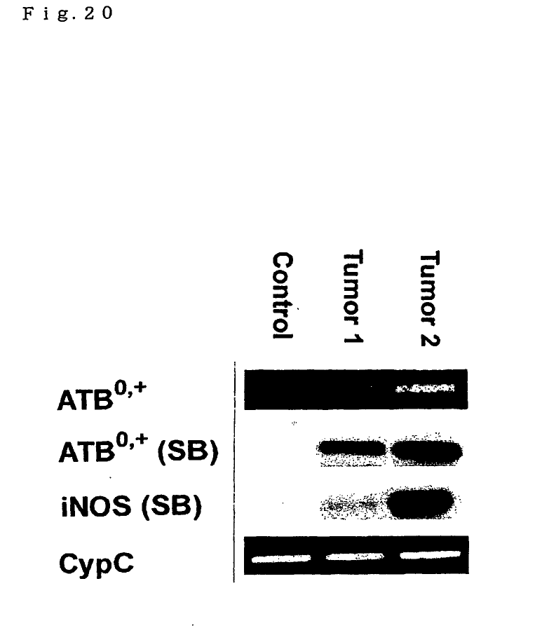

The present inventors assessed the expression of ATB^{0,+} in the tumor tissues. The present inventors used the mRNA from the mammary gland tumors of 105-day old FvB wild type polyoma Middle T antigen female mice and corresponding mammary gland from the control mice. The tumor mRNA samples were collected from two mice. The control mammary glands were collected from three mice and pooled for RNA preparation, because their mammary glands were too small to prepare mRNA for each mouse. The steady-state levels of ATB^{0,+} mRNA were examined by a semi-quantitative RT-PCR in the mammary gland tumor and the corresponding control (FIG. 20). ATB^{0,+} was not detected in control mammary gland, while in both tumor samples ATB^{0,+} was detected by RT-PCR. The present inventors quantified the induction level of ATB^{0,+} in tumor by semi-quantitative RT-PCR with southern blot. ATB^{0,+} increased approximately 58 times and 88 times. The present inventors also quantified the induction level of iNOS. iNOS also increased approximately 33 times and 134 times in both tumor samples. It is well known that many tumors have higher expression of iNOS, and iNOS may be playing an important role in oncogenesis and tumor growth-(Jaiswal, M. et al., Am. J. Physiol., 281, G626-G634, (2001), Thomsen, L. L. et al., Cancer Research, 57: 3300-3304, (1997)). The present inventors also found the higher expression of ATB^{0,+} in a human breast cancer cell line, MCF-7, and a human hepatocarcinoma cell line, Hep-G2.

Industrial Applicability

The present invention has revealed the compounds transportable by ATB^{0,+}. Based on the information about these compounds, drugs transportable by ATB^{0,+} may be designed, produced and screened. Such drugs may serve to treat and/or prevent the diseases in which NOS, phenylglycine, carnitine, D-amino acids and so forth are involved. The ATB^{0,+} gene may be administered to patients to be used for gene therapy of the diseases as described above.

1. A method for screening for a drug or prodrug having ability to be transported by ATB^{0,+}, comprising the steps of:
(a) selecting compounds having the ability to be transported by ATB^{0,+};
(b) relating the selected compounds to a disease that can be treated and/or prevented with said compounds; and
(c) selecting a compound that is related to a disease in step (b).

2. The method according to claim 1, wherein said compound having the ability to be transported by ATB^{0,+} is an NOS inhibitor, phenylglycine, carnitine or a D-amino acid, or a derivative thereof, and an amino acid-based prodrug.

3. A method for designing a compound having the ability to be transported by ATB^{0,+}, wherein said method is provided for designing a compound selected from a group consisting of NOS inhibitors, phenylglycine, carnitine and D-amino acids, and derivatives thereof, and an amino add-based prodrug.

4. A method for producing a compound having the ability to be transported by ATB^{0,+}, comprising the steps of:
(a) designing a compound selected from a group consisting of NOS inhibitors, phenylglycine, carnitine and D-amino acids, and derivatives thereof, and amino acid-based prodrugs; and
(b) synthesizing the designed compound.

5. The method according to claim 4, further comprising the step of determining whether the synthesized compound has the ability to be transported by ATB^{0,+} to select a compound to be transported.

6. A method for producing a drug containing, as an active ingredient, a compound with the ability to be transported by ATB^{0,+}, wherein said method comprises the steps of:
(a) designing a compound selected from a group consisting of NOS inhibitors, phenylglycine, carnitine and D-amino acids, and derivatives thereof, and acid-based prodrugs;
(b) synthesizing the designed compound; and
(c) determining whether the synthesized compound has the ability to be transported by ATB^{0,+} and selecting a compound to be transported.

7. A method for producing a drug having, as an active ingredient, a compound with the ability to be transported by ATB^{0,+}, wherein said method comprises the steps of:
(a) designing a compound selected from a group consisting of NOS inhibitors, phenylglycine, carnitine and D-amino acids, and derivatives thereof and amino acid-based prodrugs,
(b) synthesizing the designed compound; and
(c) relating the synthesized compound to a disease that can be treated and/or prevented with said compound.

8. A method for transport of a compound mediated by ATB^{0,+}, wherein said compound is selected from a group consisting of NOS inhibitors, phenylglycine, carnitine and D-amino acids, and derivatives thereof, and amino acid-based prodrug.

9. A method for transport of a compound mediated by ATB⁰⁺, wherein said compound is selected from a group consisting of NOS inhibitions, phenylglycine, carnitine and D-amino acids, and derivatives thereof, and amino aid-based prodrugs, wherein said compound is labeled with a radioactive substance or conjugated with toxin.

10. The method according to any one of claim 2 to 9, wherein said NOS inhibitor is a derivative of arginine, lysine, citrulline and ornithine.

11. The method according to any one of claim 2 to 9, wherein said phenylglycine derivative is a (S)-2-amino-2-(3-(N′-nitroguanidino)phenyl)acetic acid.

12. A therapeutic drug for a disease that can be treated and/or prevented with a compound selected from a group consisting of NOS inhibitors, phenylglycine, carnitine and D-amino acids, and derivatives thereof, wherein said therapeutic drug comprises the ATB^{0,+} gene as an active ingredient.

13. The therapeutic drug according to claim 12, wherein said phenylglycine derivative is a (S)-2-amino-2-(3-(N′-nitroguanidino)phenyl)acetic acid.

14. A gene therapy for a disease that can be treated and/or prevented with a compound selected from a group consisting of NOS inhibitors, phenylglycine, carnitine and D-amino acids, and derivatives thereof, wherein said method comprises the step of administering the ATB^{0,+} gene.

15. The gene therapy according to claim 14, wherein said phenylglycine derivative is a (S)-2-amino-2-(3-(N′-nitroguanidino)phenyl)acetic acid.

16. A therapeutic drug for cancer, comprising a compound having the ability to be transported by ATB^{0,+} as an active ingredient.

17. The therapeutic drug according to claim 16, wherein said cancer is iNOS expressed cancer.

18. The therapeutic drug according to claim 16, wherein said cancer is breast cancer or hepatic cancer.

19. The therapeutic drug according to claim 16, wherein said cancer is a NOS inhibitor.

20. The therapeutic drug according to claim 19, wherein said NOS inhibitor is a derivative of arginine, lysine, citrulline, ornithine and phenylglycine.

21. The therapeutic drug according to claim 16, wherein said phenylglycine derivative is a (S)-2-amino-2-(3-(N′-nitroguanidino)phenyl)acetic acid.

22. A method for treating cancer, comprising the step of administering a compound having the ability to be transported by ATB^{0,+}.

23. The method according to claim 22, wherein said cancer is iNOS expressed cancer.

24. The method according to claim 22, wherein said cancer is breast cancer or hepatic cancer.

25. The method according to claim 22, wherein said compound is a NOS inhibitor.

26. The method according to claim 25, wherein said NOS inhibitor is a derivative of arginine, lysine, citrulline, ornithine and phenylglycine.

27. The method according to claim 22, wherein said phenylglycine derivative is a (S)-2-amino-2-(3-(N′-nitroguanidino)phenyl)acetic acid.

28. The of a compound having the ability to be transported by ATB^{0,+} for producing a therapeutic drug for cancer.

29. The use according to claim 28, wherein said cancer is iNOS expressed cancer.

30. The use according to claim 28, wherein said cancer is breast cancer or hepatic cancer.

31. The use according to claim 28, wherein said compound is a NOS inhibitor.

32. The method according to claim 31, wherein said NOS inhibitor is a derivative of arginine, lysine, citrulline, ornithine and phenylglycine.

33. The method according to claim 32, wherein said phenylglycine derivative is a (S)-2-amino-2-(3-(N′-nitroguanidino)phenyl)acetic acid.

34. A therapeutic drug from inflammation, comprising a compound having the ability to be transported by ATB^{0,+} as an active ingredient.

35. The therapeutic drug according to claim 34, wherein said inflammation is sepsis.

36. The therapeutic drug according to claim 34, wherein said inflammation is inflammatory bowel disease.

37. The therapeutic drug according to claim 34, wherein said compound is a NOS inhibitor.

38. The therapeutic drug according to claim 37, wherein said NOS inhibitor is a derivative of arginine, lysine, citrulline, ornithine and phenylglycine.

39. The therapeutic drug according to claim 38, wherein said phenylglycine derivative is a (S)-2-amino-2-(3-(N′-nitroguanidino)phenyl)acetic acid.

40. A method for treating inflammation, comprising the step of administering a compound having the ability to be transported by ATB^{0,+}.

41. The method according to claim 40, wherein said inflammation is sepsis.

42. The method according to claim 40, wherein said inflammation is inflammatory bowel disease.

43. The method according to claim 40, wherein said compound is a NOS inhibitor.

44. The method according to claim 43, wherein said NOS inhibitor is a derivative of arginine, lysine, citrulline, ornithine and phenylglycine.

45. The method according to claim 44, wherein said phenylglycine derivative is a (S)-2-amino-2-(3-(N′-nitroguanidino)phenyl)acetic acid.

46. Use of a compound having the ability to be transported by ATB^{0,+}0 for producing a therapeutic drug for inflammation.

47. The use according to claim 46, wherein said inflammation is sepsis.

48. The use according to claim 46, wherein said inflammation is inflammatory bowel disease.

49. The use according to claim 46, wherein said compound is a NOS inhibitor.

50. The method according to claim 22, wherein said NOS inhibitor is a derivative of arginine, lysine, citrulline, ornithine and phenylglycine.

51. The method according to claim 22, wherein said phenylglycine derivative is a (S)-2-amino-2-(3-(N′-nitroguanidino)phenyl)acetic acid

52. A (S)-2-tert-Butoxycarbonylamino-2-(3-(N′-nitroguanidino)phenyl)acetic acid tert-butyl ester.

53. A (S)-2-amino-2-(3-(N′-nitroguanidino)phenyl)acetic acid.
